(19) United States
(12) Patent Application Publication
(10) Pub. No.: US 2023/0143486 A1
(43) Pub. Date: May 11, 2023

(54) METHODS OF GENERATING MIDBRAIN DOPAMINE NEURONS, MIDBRAIN NEURONS AND USES THEREOF

(71) Applicant: MEMORIAL SLOAN-KETTERING CANCER CENTER, NEW YORK, NY (US)

(72) Inventors: Lorenz STUDER, New York, NY (US); Tae-Wan KIM, New York, NY (US); So-Yeon KOO, New York, NY (US)

(73) Assignee: MEMORIAL SLOAN-KETTERING CANCER CENTER, NEW YORK, NY (US)

(21) Appl. No.: 17/956,957

(22) Filed: Sep. 30, 2022

Related U.S. Application Data
(63) Continuation of application No. PCT/US2021/025596, filed on Apr. 2, 2021.
(60) Provisional application No. 63/004,138, filed on Apr. 2, 2020.

(51) Int. Cl. C12N 5/0793; A61P 25/16; A61K 35/30

(52) U.S. Cl. CPC C12N 5/0619; A61P 25/16; A61K 35/30; C12N 2501/113; C12N 2501/41; C12N 2501/415

(57) ABSTRACT

The present disclosure provides methods for generating midbrain dopamine neurons and precursors thereof, midbrain dopamine neurons and precursors thereof generated by such methods and compositions comprising such cells, and uses thereof for preventing, modeling, and/or treating a neurological disorder.

### CROSS-REFERENCE TO RELATED APPLICATIONS

The present application is a continuation of International Patent Application No. PCT/US2021/025596, filed on Apr. 2, 2021, which claims priority to U.S. Provisional Application No. 63/004,138, filed on Apr. 2, 2020, the content of each of which is incorporated by reference in its entirety herein, and to each of which priority is claimed.

### 1. INTRODUCTION

The present disclosure provides methods for generating midbrain dopamine (mDA) neurons and precursors thereof, mDA neurons and precursors thereof generated by such methods and composition comprising such cells. The present disclosure also provides uses of the mDA neurons and composition comprising thereof for preventing, modeling, and/or treating neurological disorders.

### 2. BACKGROUND

Parkinson's disease (PD) is characterized by the loss of mDA neurons that lead to well-known motor symptoms such as tremor, rigor, and bradykinesia (Lees, et al. Lancet 373, 2055-2066 (2009)). While other cell types such as enteric, olfactory or cortical neurons are also affected (Del Tredici, et al. Neuropathol Appl Neurobiol 42, 33-50 (2016)), mDA neurons remain the key focus for developing novel cell-based treatments (Barker, et al. Nature reviews Neurology 11, 492-503 (2015); Tabar, et al. Nat Rev Genet 15, 82-92 (2014)) and for PD disease modeling (Sanchez-Danes, et al. EMBO Mol Med 4, 380-395 (2012); Miller, et al. Cell stem cell 13, 691-705 (2013); Chung, et al. Stem Cell Reports 7, 664-677 (2016); Reinhardt, et al. Cell stem cell 12, 354-367 (2013); Chung, et al. Science 342, 983-987 (2013); Cooper, et al. Sci Transl Med 4, 141ra190 (2012)). Human pluripotent stem cells (hPSCs), comprising both human ES and iPS cells, have become the cell type of choice for deriving mDA neurons in vitro. Despite progress in human mDA derivation, there is a need for novel protocols. For cell therapy, there is still no clear agreement on the optimal type and stage of mDA neurons to be used and considerable molecular and functional differences in the behavior of hPSC-derived versus (vs) primary fetal DA neurons have been reported in vitro (La Manno, et al. Cell 167, 566-580 e519 (2016)) and in vivo (Tiklova, et al. Nature communications 10, 581 (2019). Furthermore, there are no reliable cell purification strategies and cell survival of hPSC-derived mDA neurons remains low (˜10% of grafted cells) (Sanchez-Danes, et al. EMBO Mol Med 4, 380-395 (2012)). Low mDA survival could cause variability in clinical cell dosing and complicates the routine application of this technology for the broader PD community. These questions are important for both cell therapy and disease modeling applications.

In human disease modeling, the variation in mDA neuron yield and purity across hPSC lines introduces noise for detecting disease-related phenotypes and complicates drug discovery efforts. Access to defined mDA neuron subtypes would enable studies on the mechanism of cell-type specific vulnerability in PD (Surmeier, et al. Cold Spring Harb Perspect Med 2, a009290 (2012); Anderegg, et al. FEBS Lett 589, 3714-3726 (2015); Chung, et al. Hum Mol Genet 14, 1709-1725 (2005); Brichta and Greengard. Front Neuroanat 8, 152 (2014)). Having access to defined, more robust mDA neuron cultures and the possibility of driving mDA neurons subtypes will greatly accelerate efforts in PD disease modeling and may allow for improved products for mDA neuron cell therapy in the future.

Therefore, there is still a need for improved methods for generating mDA neurons that have improved in vivo survival and are suitable for treating neurological disorders such as Parkinson's disease.

### 3. SUMMARY OF THE INVENTION

The present disclosure provides methods for generating mDA neurons and precursors thereof, mDA neurons and precursors thereof generated by such methods, compositions comprising such cells, and uses of such cells and compositions for preventing and/or treating neurological disorders.

The present disclosure provides in vitro methods for inducing differentiation of stem cells. In certain embodiments, the method comprises: contacting the stem cells with at least one inhibitor of Small Mothers Against Decapentaplegic (SMAD) signaling, at least one activator of Sonic hedgehog (SHH) signaling, and at least one activator of wingless (Wnt) signaling; and contacting the cells with at least one activator of fibroblast growth factor (FGF) signaling and at least one inhibitor of Wnt signaling to obtain a population of differentiated cells expressing at least one marker indicating a midbrain dopamine neuron (mDA) or a precursor thereof.

In certain embodiments, the contact of the cells with the at least one inhibitor of Wnt signaling is initiated at least about 5 days from the initial contact of the stem cells with the at least one inhibitor of SMAD signaling. In certain embodiments, the contact of the cells with the at least one inhibitor of Wnt signaling is initiated no later than about 15 days from the initial contact of the stem cells with the at least one inhibitor of SMAD signaling. In certain embodiments, the contact of the cells with the at least one inhibitor of Wnt signaling is initiated about 10 days from the initial contact of the stem cells with the at least one inhibitor of SMAD signaling. In certain embodiments, the contact of the cells with the at least one inhibitor of Wnt signaling is initiated 10 days, 11 days, 12 days, or 13 days from the initial contact of the stem cells with the at least one inhibitor of SMAD signaling.

In certain embodiments, the cells are contacted with the at least one inhibitor of Wnt signaling for at least about 1 day. In certain embodiments, the cells are contacted with the at least one inhibitor of Wnt signaling for up to about 30 days or up to about 25 days. In certain embodiments, the cells are contacted with the at least one inhibitor of Wnt signaling for about 5 days, about 15 days, or about 20 days. In certain embodiments, the cells are contacted with the at least one inhibitor of Wnt signaling for 4 days, 5 days, 6 days, 7 days, 14 days, 15 days, 19 days, or 20 days.

In certain embodiments, the contact of the cells with the at least one activator of FGF signaling is initiated at least about 5 days from the initial contact of the cells with the at least one inhibitor of SMAD signaling. In certain embodiments, the contact of the cells with the at least one activator of FGF signaling is initiated at least about 10 days from the initial contact of the cells with the at least one inhibitor of SMAD signaling. In certain embodiments, the contact of the cells with the at least one activator of FGF signaling is initiated no later than about 20 days from the initial contact of the cells with the at least one inhibitor of SMAD signaling. In certain embodiments, the contact of the cells with the at least one activator of FGF signaling is initiated no later than 18 days from the initial contact of the cells with the at least one inhibitor of SMAD signaling. In certain embodiments, the contact of the cells with the at least one activator of FGF signaling is initiated no later than the majority of the midbrain dopamine neuron precursors has differentiated into postmitotic neurons. In certain embodiments, the contact of the cells with the at least one activator of FGF signaling is initiated about 10 days from the initial contact of the cells with the at least one inhibitor of SMAD signaling. In certain embodiments, the contact of the cells with the at least one activator of FGF signaling is initiated 10 days, 11 days, 12 days, or 13 days from the initial contact of the cells with the at least one inhibitor of SMAD signaling. In certain embodiments, the cells are contacted with the at least one activator of FGF signaling for at least about 1 day, and/or for up to about 20 days. In certain embodiments, the cells are contacted with the at least one activator of FGF signaling for at least about 3 days, and/or for up to about 10 days. In certain embodiments, the cells are contacted with the at least one activator of FGF signaling for at least 4 days, and/or for up to 7 days. In certain embodiments, the cells are contacted with the at least one activator of FGF signaling for about 5 days. In certain embodiments, the cells are contacted with the at least one activator of FGF signaling for 4 days, 5 days, 6 days, or 7 days.

In certain embodiments, the cells are contacted with the at least one inhibitor of SMAD signaling for about 5 days. In certain embodiments, the cells are contacted with the at least one inhibitor of SMAD signaling for 6 days or 7 days.

In certain embodiments, the cells are contacted with the at least one activator of SHH signaling for about 5 days. In certain embodiments, the cells are contacted with the at least one activator of SHH signaling for 6 days or 7 days.

In certain embodiments, the cells are contacted with the at least one activator of Wnt signaling for about 15 days. In certain embodiments, the cells are contacted with the at least one activator of Wnt signaling for 16 days or 17 days. In certain embodiments, the concentration of the at least one activator of Wnt signaling is increased about 4 days from its initial contact with the stem cells. In certain embodiments, the concentration of the at least one activator of Wnt signaling is increased by between about 200% and about 1000% from the initial concentration of the at least one activator of Wnt signaling. In certain embodiments, the concentration of the at least one activator of Wnt signaling is increased by about 500% from the initial concentration of the at least one activator of Wnt signaling. In certain embodiments, the concentration of the at least one activator of Wnt signaling is increased to from about 1 μM to between about 5 μM and about 10 μM. In certain embodiments, the concentration of the at least one activator of Wnt signaling is increased to a concentration of about 6 μM.

In certain embodiments, the at least one inhibitor of Wnt signaling is capable of inhibiting canonical Wnt signaling. In certain embodiments, the at least one inhibitor of Wnt signaling is capable of inhibiting non-canonical Wnt signaling and canonical Wnt signaling. In certain embodiments, the at least one inhibitor of Wnt signaling is selected from the group consisting of IWP2, IWR1-endo, XAV939, IWP-O1, IWP12, Wnt-C59, IWP-L6, ICG-001, LGK-974, IWR-1, ETC-159, iCRT3, IWP-4, Salinomycin, Pyrvinium Pamoate, iCRT14, FH535, CCT251545, KYA1797K, Wogonin, NCB-0846, Hexachrorophene, PNU-74654, KY02111, S03031 (KY01-I), S02031 (KY02-I), Triptonide, BC2059, PKF115-584, Quercetin, NSC668036, G007-LK, MSAB, LF3, JW55, Isoquercitrin, WIKI4, derivatives thereof, and combinations thereof. In certain embodiments, the at least one inhibitor of Wnt signaling is selected from the group consisting of IWP2, IWR1-endo, IWP-O1, IWP12, Wnt-C59, IWP-L6, LGK-974, IWR-1, ETC-159, iCRT3, IWP-4, Salinomycin, Pyrvinium Pamoate, iCRT14, FH535, CCT251545, Wogonin, NCB-0846, Hexachrorophene, KY02111, S03031 (KY01-I), S02031 (KY02-I), BC2059, PKF115-584, Quercetin, NSC668036, G007-LK, derivatives thereof, and combinations thereof. In certain embodiments, the at least one inhibitor of Wnt signaling is selected from the group consisting of XAV939, ICG-001, PNU-74654, Triptonide, KYA1797K, MSAB, LF3, JW55, Isoquercitrin, WIKI4, derivatives thereof, and combinations thereof. In certain embodiments, the at least one inhibitor of Wnt signaling comprises IWP2.

In certain embodiments, the at least one activator of FGF signaling is selected from the group consisting of FGF18, FGF17, FGF8a, FGF8b, FGF4, FGF2, and combination thereof. In certain embodiments, the at least one activator of FGF signaling is capable of causing expansion of the midbrain and upregulating midbrain gene expression. In certain embodiments, the at least one activator of FGF signaling is selected from the group consisting of FGF18, FGF17, FGF8a, FGF4, FGF2, and combination thereof. In certain embodiments, the at least one activator of FGF signaling comprises FGF18.

In certain embodiments, the at least one inhibitor of SMAD signaling comprises an inhibitor of TGFβ/Activin-Nodal signaling, an inhibitor of bone morphogenetic protein (BMP) signaling, or a combination thereof. In certain embodiments, the at least one inhibitor of TGFβ/Activin-Nodal signaling comprises an inhibitor of ALK5. In certain embodiments, the at least one inhibitor of TGFβ/Activin-Nodal signaling is selected from the group consisting of SB431542, derivatives of SB431542, and combinations thereof. In certain embodiments, the derivative of SB431542 comprises A83-01. In certain embodiments, the at least one inhibitor of TGFβ/Activin-Nodal signaling comprises SB431542. In certain embodiments, the at least one inhibitor of BMP signaling is selected from the group consisting of LDN193189, Noggin, dorsomorphin, derivatives of LDN193189, derivatives of Noggin, derivatives of dorsomorphin, and combinations thereof. In certain embodiments, the at least one inhibitor of BMP comprises LDN-193189.

In certain embodiments, the at least one activator of Wnt signaling comprises an inhibitor of glycogen synthase kinase 3β (GSK3β) signaling. In certain embodiments, the at least one activator of Wnt signaling is selected from the group consisting of CHIR99021, CHIR98014, AMBMP hydrochloride, LP 922056, Lithium, deoxycholic acid, BIO, SB-216763, Wnt3A, Wnt1, Wnt5a, derivatives thereof, and combinations thereof. In certain embodiments, the at least one activator of Wnt signaling comprises CHIR99021.

In certain embodiments, the at least one activator of SHH signaling is selected from the group consisting of SHH proteins, Smoothened agonists (SAG), and combinations thereof. In certain embodiments, the SHH protein is selected from the group consisting of recombinant SHHs, modified N-terminal SHHs, and combinations thereof. In certain embodiments, the modified N-terminal SHH comprises two Isoleucines at the N-terminus. In certain embodiments, the modified N-terminal SHH has at least about 90% sequence identity to an un-modified N-terminal SHH. In certain embodiments, the un-modified N-terminal SHH is a un-modified mouse N-terminal SHH or a un-modified human N-terminal SHH. In certain embodiments, the modified N-terminal SHH comprises SHH C25II. In certain embodiments, the SAG comprises purmorphamine.

In certain embodiments, at least about 80% of the differentiated cells express FOXA2 and EN1 about 15 days from the initial contact of the stem cells with the at least one inhibitor of SMAD signaling. In certain embodiments, greater than about 80% or greater than about 90% of the differentiated cells express FOXA2 and EN1 16 days from the initial contact of the stem cells with the at least one inhibitor of SMAD signaling.

In certain embodiments, the at least one marker indicating a midbrain dopamine neuron or a precursor thereof is selected from the group consisting of EN1, OTX2, TH, NURR1, FOXA2, PITX3, LMX1A, LMO3, SNCA, ADCAP1, CHRNA4, GIRK2, ALDH1A1, SOX6, WNT1, VMAT2, DAT (SLC6A3), and combinations thereof. In certain embodiments, the differentiated cells do no express at least one marker selected from the group consisting of PAX6, EMX2, LHX2, SMA, SIX1, PITX2, SIM1, POU4F1, PHOX2A, BARHL1, BARHL2, GBX2, HOXA1, HOXA2, HOXB1, HOXB2, POUSF1, NANOG, and combinations thereof.

In certain embodiments, the method further comprises isolating cells that express at least one positive surface marker and do not express at least one negative surface marker. In certain embodiments, the at least one positive surface marker is selected from the group consisting of CD171, CD184, and combinations thereof. In certain embodiments, the at least one positive surface marker comprises CD184. In certain embodiments, the at least one negative surface marker is selected from CD49e, CD99, CD340, and combinations thereof. In certain embodiments, the at least one negative surface marker comprises CD49e. In certain embodiments, the method comprises sorting cells that express CD184 and do not express CD49e.

In certain embodiments, the stem cells are pluripotent stem cells. In certain embodiments, the stem cells are selected from the group consisting of nonembryonic stem cells, embryonic stem cells, induced pluripotent stem cells, and combinations thereof. In certain embodiments, the stem cells are human stem cells, non-human primate stem cells, or rodent stem cells. In certain embodiments, the stem cells are human stem cells.

The present disclosure provides cell populations of in vitro differentiated cells, wherein the in vitro differentiated cells are obtained by a differentiation method disclosed herein.

The present disclosure further provides compositions comprising the cell populations disclosed herein. In certain embodiments, the composition is a pharmaceutical composition further comprising a pharmaceutically acceptable carrier.

Furthermore, the present disclosure provides kits for inducing differentiation of stem cells to midbrain dopamine neurons or precursors thereof. In certain embodiments, the kit comprises (a) at least one inhibitor of SMAD signaling, (b) at least one activator of SHH signaling; (c) at least one activator of Wnt signaling; (d) at least one inhibitor of Wnt signaling; and (e) at least one activator of FGF signaling. In certain embodiments, the kit further comprises (f) instructions for inducing differentiation of the stem cells into a population of differentiated cells that express at least one marker indicating a midbrain dopamine neuron or a precursor thereof.

The present disclosure further provides methods of preventing, modeling, and/or treating a neurological disorder in a subject. In certain embodiments, the method comprises administering to the subject an effective amount of the cell population disclosed herein or the composition disclosed herein. The cell population disclosed herein or the composition disclosed herein can be used in preventing, modeling, and/or treating a neurological disorder in a subject. In certain embodiments, the neurological disorder is characterized by reduction of midbrain dopamine neuron function. In certain embodiments, the reduction of midbrain dopamine neuron function is age related. In certain embodiments, the neurological disorder is selected from the group consisting of Parkinsonism, Parkinson's disease, Huntington's disease, Alzheimer's disease, multiple sclerosis, and combinations thereof. In certain embodiments, the neurological disorder is selected from the group consisting of Parkinsonism, Parkinson's disease, and combinations thereof. In certain embodiments, the symptom for a neurological disorder is selected from the group consisting of tremor, bradykinesia, flexed posture, postural instability, rigidity, dysphagia, and dementia.

### 4. BRIEF DESCRIPTION OF THE DRAWINGS

FIG. 1 shows the effects of Wnt signaling on ALDH1A1 induction in mDA cells differentiated using different protocols. The mRNA expression levels of FOXA2, LMX1A, EN1, WNT1, OTX2, ALDH1A1 and PAX6 were evaluated in day 16-differentiated mDA cells produced using Wnt-boost, Wnt-boost+IWP2 (day 10-day 16), or Wnt-boost+IWP2 (day 12-day 16) protocols, with or without FGF18. SMA mRNA expression level was not detectable.

FIGS. 2A and 2B show that Wnt boost protocol in combination with FGF18 and IWP2 generated optimal A/P and DN patterned mDA precursors. FIG. 2A shows FACS analysis of day 16-differentiated mDA precursors using different protocols. Cells were stained with anti-EN1 and anti-FOXA2 antibodies. FIG. 2B shows immune-staining images of day 16-differentiated mDA.

FIG. 3 shows the effect of IWP2 on the expression of marker genes in the differentiated cells. The mRNA expression levels of FOXA2, LMX1A, OTX2, EN1, ALDH1A1, BARHL2, BARHL1, PAX6, ALDH2, and WNT1 were measured in day 16-differentaited cells produced using Wnt-boost protocol, with or without the addition of FGF18 and/or IWP2 from day 12 to day 16.

FIG. 4 shows the effect of IWP2 on the expression of marker genes in the differentiated cells. The mRNA expression levels of FOXA2, LMX1A, OTX2, EN1, ALDH1A1, PAX6 and PITX3 were evaluated in day-40 differentiated cells produced using Wnt-boost protocol, with or without the addition of FGF18 and/or IWP2 from day 12 to day 16.

FIG. 5 shows the gating paradigm of the double sorting strategy. Differentiated mDA cells were sorted based on the expression of CD49e and CD184 marker proteins.

FIG. 6 shows the morphology of sorted day 40-differentiated CD49^{weak}/CD184^{weak} cells and CD49^{weak}/CD184^{strong} cells. Cells were sorted on day 25 of in vitro differentiation under Wnt-boost or Wnt-boost+FGF18/IWP2 (day 12-day 16) protocols.

FIG. 7 shows the mRNA expression of dopamine neuron marker genes in sorted day 40-differentiated CD49^{weak}/CD184^{weak} cells and CD49^{weak}/CD184^{strong} cells. Cells were sorted on day 25 of in vitro differentiation under Wnt-boost or Wnt-boost+FGF18/IWP2 (day 12-day 16) protocols.

FIG. 8 shows the mRNA expression of non-dopamine neuron marker genes in sorted day 40-differentiated CD49^{weak}/CD184^{weak} cells and CD49^{weak}/CD184^{strong} cells. Cells were sorted on day 25 of in vitro differentiation under Wnt-boost or Wnt-boost+FGF18/IWP2 (day 12-day 16) protocols.

FIGS. 9A-9C show representative immuno-staining images of sorted day 40-differentiated CD49^{weak}/CD184^{weak} cells and CD49^{weak}/CD184^{strong} cells. FIG. 9A shows the expression of FOXA2, TH, and MAP2. FIG. 9B shows the expression of ALDH1A1, EN1, and TH. FIG. 9 C shows the expression of ALDH1A1, EN1, and TH. Cells were sorted on day 25 of in vitro differentiation under Wnt-boost or Wnt-boost+FGF18/IWP2 (day 12-day 16) protocols.

FIGS. 10A-10B show representative immune-staining images of differentiated cells after the cells were transplanted in mouse. Differentiated cells were obtained using Wnt-boost or Wnt-boost+FGF18/IWP2 (day 12-day 16) protocols, and were transplanted in mice. Grafted cells were immune-stained one month after transplantation. The expression of hNCAM, TH, and ALDH1A1 were evaluated (FIG. 10A). The expression of Ki67 was also evaluated (FIG. 10B).

FIGS. 11A-11C show representative immune-staining images of differentiated cells after the cells were transplanted in mouse. Differentiated cells were obtained using Wnt-boost or Wnt-boost+FGF18/IWP2 (day 12-day 16) protocols, and were transplanted in mice. Grafted cells were immune-stained one months after transplant. FIG. 11A shows expression of SC121. FIG. 11B shows the expression of TH and Nurr1-GFP. FIG. 11C shows the expression of ALDH1A1 and SOX6-RFP.

FIG. 12 shows the effect of IWP2 on the expression of marker genes in differentiated cells. The mRNA expression levels of marker genes were measured in day 16-differentiated cells produced using Wnt-boost protocol, with or without the addition of FGF18 and/or IWP2 from day 12 to day 16.

FIG. 13 shows the effect of IWP2 on the expression of marker genes in the differentiated cells. The mRNA expression levels of various genes were evaluated in day 40-differentiated cells produced using Wnt-boost protocol, with or without the addition of FGF18 and/or IWP2 from day 12 to day 16.

FIGS. 14A and 14B show representative images of immunofluorescence staining of day 60-differentiated cells produced using Wnt-boost protocol, with or without the addition of FGF18 and/or IWP2 from day 12 to day 16. FIG. 14A shows immune-staining images of day 60 differentiated cells that express FOXA2, TH, and MAP2 for each condition. FIG. 5B shows different staining panels marking EN1 and TH, showing differential expression of EN1 among TH⁺ dopamine neurons at day 60 is.

FIG. 15 shows the mRNA expression of marker genes in sorted day 40-differentiated CD49^{weak}/CD184^{weak} cells and CD49^{weak}/CD184^{strong} cells. Cells were sorted on day 25 of in vitro differentiation under Wnt-boost or Wnt-boost+FGF18/IWP2 (day 12-day 16) protocols.

FIG. 16 shows the mRNA expression of marker genes in sorted day 40-differentiated CD49^{weak}/CD184^{weak} cells and CD49^{weak}/CD184^{strong} cells. Cells were sorted on day 25 of in vitro differentiation under Wnt-boost or Wnt-boost+FGF18/IWP2 (day 12-day 16) protocols.

FIG. 17 shows the representative immuno-staining images of sorted day 60-differentiated CD49^{weak}/CD184^{weak} cells and CD49^{weak}/CD184^{strong} cells. Cells were sorted on day 25 of in vitro differentiation under Wnt-boost or Wnt-boost+FGF18/IWP2 (day 12-day 16) protocols.

FIG. 18 shows the representative immuno-staining images of sorted day 60-differentiated CD49^{weak}/CD184^{strong} cells. Cells were sorted on day 25 of in vitro differentiation under Wnt-boost or Wnt-boost+FGF18/IWP2 (day 12-day 16) protocols.

FIG. 19 shows the mRNA expression of marker genes in day 30-differentiated cells produced using Wnt-boost protocol with or without the addition of IWP2 and FGF18 from day 12 to day 16, and with or without the addition of IWP2 from day 17 to day 30.

FIG. 20 shows FACS-mediated sorting of day 25-differentiated cells produced from Wnt-boost protocol with or without the addition of IWP2 from day 12 to day 25, or from day 12 to day 16, and with or without the addition of FGF18 from day 12 to day 16.

FIG. 21 shows the mRNA expression of marker genes in sorted day 28-differentiated CD49^{weak}/CD184^{weak} cells and CD49^{weak}/CD184^{strong} cells. Cells were sorted on day 25 of in vitro differentiation under Wnt boost with or without the addition of IWP2 from day 12 to day 25, or from day 12 to day 16, and with or without the addition of FGF18 from day 12 to day 16.

FIG. 22 shows the mRNA expression of non-dopamine neuron marker genes in sorted day 28-differentiated CD49^{weak}/CD184^{weak} cells and CD49^{weak}/CD184^{strong} cells. Cells were sorted on day 25 of in vitro differentiation under Wnt boost with or without the addition of IWP2 from day 12 to day 25, or from day 12 to day 16, and with or without the addition of FGF18 from day 12 to day 16.

FIG. 23 shows the immuno-staining images of sorted day 28-differentiated CD49^{weak}/CD184^{weak} cells and CD49^{weak}/CD184^{strong} cells. Cells were sorted on day 25 of in vitro differentiation under Wnt boost with or without the addition of IWP2 from day 12 to day 25, or from day 12 to day 16, and with or without the addition of FGF18 from day 12 to day 16.

FIG. 24 shows representative immune-staining images of differentiated cells after the cells were transplanted in mouse. Differentiated cells were obtained using Wnt-boost or Wnt-boost+FGF18/IWP2 (day 12-day 16) protocols, and were transplanted in mice. Grafted cells were immune-stained one month after transplantation. The expression of FOXA2, SC121, ALDH1A1, EN1 and Ki67 were evaluated.

FIG. 25 shows representative immune-staining images of differentiated cells after the cells were transplanted in mouse brain. Differentiated cells were obtained using Wnt-boost or Wnt-boost+FGF18/IWP2 (day 12-day 16) protocols, and were transplanted in mice. Grafted cells were immune-stained one month after transplantation and evaluated for any proliferating cells marked by Ki67.

FIG. 26 shows representative immune-staining images of the mouse intrastriatal graft of the frozen batch of the dopamine precursors (day 16 differentiated). Day 16-differentiated cells were obtained using Wnt-boost+FGF18/IWP2 (day 12-day 16) protocol, and were frozen using controlled-rate freezing machine. Frozen cells were thawed and directly transplanted in the striatum of the NOD-SCID mice. Grafted cells were immune-stained one month after transplantation.

FIG. 27 shows representative immune-staining images of differentiated cells after the cells were transplanted in mouse. Differentiated cells were obtained using Wnt-boost+FGF18/IWP2 (day 12-day 16) protocols, and were transplanted in mice. Grafted cells were immune-stained 4 months after transplantation.

FIG. 28 shows representative immune-staining images of differentiated cells after the sorted CD49^{weak}/CD184^{strong} cells were transplanted in mouse. Cells were sorted on day 25 of in vitro differentiation under Wnt-boost or Wnt-boost+FGF18/IWP2 (day 12-day 16) protocols, and were transplanted in mice. Grafted cells were immune-stained one month after transplantation.

FIG. 29 shows representative RNA fluorescent in-situ (FISH) images of PITX3 and NURR1 among TH positive cells. mRNA signal was measured in dots within a cell, and the number of puncta was quantified at day 35, day 59, and day 82 during differentiation Differentiated cells were obtained using Wnt-boost+FGF18/IWP2 (day 12-day 16) protocols.

### 5. DETAILED DESCRIPTION

The present disclosure provides methods for generating mDA neurons and precursors thereof, mDA neurons and precursors thereof generated by such methods, compositions comprising such cells, and uses thereof for preventing and/or treating neurological disorders.

Wnt signaling is crucial for mDA neuron specification. The inventors' prior studies show that Wnt-boosting leads to robust induction of EN1 and suppression of both hindbrain and subthalamic and forebrain fates. See e.g., the Wnt-boosting methods disclosed in WO2016/196661, which is incorporated by reference in its entirety. However, protracted Wnt signaling may interfere with mDA neuron differentiation and subtype specification. In addition, the expressions of PITX3 and ALDH1A1 are at suboptimal levels in prior differentiation protocols. The present disclosure is based on the discovery that treatment with a Wnt inhibitor can improve mDA neuron derivation. In addition, such treatment with the Wnt inhibitor does not negatively impact the expression of EN1 and other mDA neuron markers, e.g., the differentiation methods disclosed herein including a Wnt inhibitor lead to sustained expression of EN1 and other mDA neuron markers. Furthermore, the treatment with the Wnt inhibitor does not increase the emergence of contaminating markers (non-mDA neuron markers).

The present disclosure is also based on the discovery that the Wnt inhibitor treatment leads to better segregation of A9 subtype neurons and A10 subtype neurons. In certain embodiments, the Wnt inhibitor treatment impacts (e.g., increases) the mRNA expression of markers indicating A9 subtype mDAs (e.g., ALDH1A1). Non-limiting examples of markers indicating A9 subtype neurons include LMO3, ALDH1A1, SOX6, VGLUT2, and NDNF. In certain embodiments, the Wnt inhibitor treatment increases number of ALDH1A1⁺ cells in vitro and in vivo (among the EN1⁺) cells. ALDH1A1 expression can be high without EN1 co-expression, and the ALDH1A1⁺EN1⁻ cells are not necessarily A9 subtype neurons and are not clearly defined cells. The differentiation methods disclosed herein including the Wnt inhibitor treatment result in high generation of cells expressing both ALDH1A1 and EN1 in vitro and in vivo after graft. In certain embodiments, the Wnt inhibitor treatment further impacts (e.g., increases) the mRNA expression of markers indicating A10 subtype mDAs. Non-limiting examples of markers indicating A10 subtype neurons include CALB1, CALB2, OTX2, CCK, VGAT (Slc32a1), and VIP. Increased mRNA expressions of A9 and A10 subtype markers support for proper specification of A9 and A10 subtype neurons. For example, certain A10 subtype neuron markers (e.g., CALB1 and CALB2) can only be seen once the cells are properly specified to exhibit A9 or A10 identity.

Furthermore, the Wnt inhibitor treatment can reduce proliferation and increase expressions of mDA neuron maturity markers. Non-limiting examples of mDA neuron maturity markers include DAT, VMAT2, PITX3, CHRNA6, and CHRNB3.

In addition, the Wnt inhibitor treatment can improve differentiation and reduce remaining Ki67⁺ proliferating cells, which can lead to improved safety profile of DA neurons.

Furthermore, grafted dopamine neurons derived from stem cells by the differentiation methods disclosed herein (e.g., including the Wnt inhibitor treatment) have improved fiber outgrowth, particularly the ALDH1A1 fibers that are expected to trigger functional recovery most efficiently.

Furthermore, the present disclosure is based on the discovery that the mDAs and precursors thereof generated by the presently disclosed methods have improved in vivo survival rate, e.g., can survive months or even years post in vivo transplantation.

The present disclosure provides improved protocols for neural induction and mDA neuron differentiation from stem cells (e.g., human pluripotent stem cells (hPSCs)), including clinical grade protocols on the verge of human use. Having access to improved mDA neuron differentiation protocols enables the field to use lower cell numbers, achieve more complete mDA neuron restoration and reduce potential side effects. Accordingly, the presently disclosed protocols improve safety, as the effect of contaminating cell types in grafts remain unclear. Finally, the presently disclosed protocols enhance the precision and reproducibility of mDA neurons in modeling human disease in a dish. The presently disclosed protocols improve faithfulness and robustness of mDA differentiation. The presently disclosed protocols can be widely adapted and can be of broad use.

Non-limiting embodiments of the presently disclosed subject matter are described by the present specification and Examples.

For purposes of clarity of disclosure and not by way of limitation, the detailed description is divided into the following subsections:
5.1. Definitions;
5.2. Methods of Differentiating Stem Cells;
5.3. Cell Populations and Compositions;
5.4. Methods of Preventing, Modeling, and/or Treating Neurological Disorders; and
5.5. Kits.

### 5.1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, a Wingless (Wnt) complex protein, including beta-catenin, NOTCH, transforming growth factor beta (TGFP), Activin, Nodal, glycogen synthase kinase 3β (GSK3β) proteins, bone morphogenetic proteins (BMP) and fibroblast growth factors (FGF). For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TFGP), Activin, Nodal, bone morphogenic proteins (BMPs), etc.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway (e.g., Wnt signaling pathway, and SMAD signaling). An inhibitor can be any compound or molecule that changes any activity of a named protein (signaling molecule, any molecule involved with the named signaling molecule, a named associated molecule, such as a glycogen synthase kinase 3β (GSK3β)). (e.g., including, but not limited to, the signaling molecules described herein). For example, an inhibitor of SMAD signaling can function, for example, via directly contacting SMAD, contacting SMAD mRNA, causing conformational changes of SMAD, decreasing SMAD protein levels, or interfering with SMAD interactions with signaling partners, and affecting the expression of SMAD target genes.

Inhibitors also include molecules that indirectly regulate biological activity, for example, SMAD biological activity, by intercepting upstream signaling molecules (e.g., within the extracellular domain, examples of a signaling molecule and an effect include: Noggin which sequesters bone morphogenic proteins, inhibiting activation of ALK receptors 1,2,3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules). Antibodies that block upstream or downstream proteins are contemplated for use to neutralize extracellular activators of protein signaling, and the like. Although the foregoing example relates to SMAD signaling inhibition, similar or analogous mechanisms can be used to inhibit other signaling molecules. Examples of inhibitors include, but are not limited to: LDN193189 (LDN) and SB431542 (SB) (LSB) for SMAD signaling inhibition, and IWP2 for Wnt inhibition. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators,” as used herein, refer to compounds that increase, induce, stimulate, activate, facilitate, or enhance activation the signaling function of the molecule or pathway, e.g., Wnt signaling, SHH signaling, etc.

As used herein, the term “Wnt” or “wingless” in reference to a ligand refers to a group of secreted proteins (e.g., integration 1 in humans) that are capable of interacting with a Wnt receptor, such as a receptor in the Frizzled and LRPDerailed/RYK receptor family. As used herein, the term “a Wnt or wingless signaling pathway refers to a signaling pathway composed of Wnt family ligands and Wnt family receptors, such as Frizzled and LRPDerailed/RYK receptors, mediated with or without β-catenin. The Wnt signaling pathway include canonical Wnt signaling (e.g., mediation by β-catenin) and non-canonical Wnt signaling (mediation without β-catenin).

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of midbrain DA precursors, or a population of undifferentiated stem cells, e.g., a population of A9 subtype midbrain dopamine neurons. Alternatively, the population may comprise more than one cell type, for example a mixed cell population, e.g., a cell population mixed of A9 subtype midbrain dopamine neurons and A10 subtype midbrain dopamine neurons.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “pluripotent” refers to an ability to develop into the three developmental germ layers of the organism including endoderm, mesoderm, and ectoderm.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “neuron” refers to a nerve cell, the principal functional units of the nervous system. A neuron consists of a cell body and its processes—an axon and at least one dendrite. Neurons transmit information to other neurons or cells by releasing neurotransmitters at synapses.

As used herein, the term “differentiation” refers to a process whereby an unspecialized embryonic cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as midbrain dopamine neurons or precursors thereof. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein marker of mDA neurons or precursors thereof, such as EN1, OTX2, TH, NURR1, FOXA2, LMX1A, PITX3, LMO3, SNCA, ADCAP1, CHRNA4, ALDH1A1, SOX6, WNT1, DAT, VMAT2, and GIRK2).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

### 5.2. Method of Differentiating Stem Cells

The present disclosure provides methods for inducing differentiation of stem cells, comprising contacting stem cells with at least one inhibitor of Small Mothers Against Decapentaplegic (SMAD) signaling (referred to as “SMAD inhibitor”), at least one activator of Sonic hedgehog (SHH) signaling (referred to as “SHH activator”), and at least one activator of wingless (Wnt) signaling (referred to as “Wnt activator”); and contacting the cells with at least one activator of fibroblast growth factor (FGF) signaling (referred to as “FGF activator”) and at least one inhibitor of Wnt signaling, to obtain a cell population comprising differentiated cells expressing at least one marker indicating a mDA neuron or a precursor thereof.

Use of an inhibitor of Wnt signaling can improve mDA neuron derivation, e.g., allowing the derivation of a broader set of mDA neurons. Protracted Wnt signaling may interfere with mDA neuron differentiation and subtype specification (Andersson, et al., Proceedings of the National Academy of Sciences of the United States of America (2013); 110, E602-610). Inhibition of Wnt signaling, e.g., by using an inhibitor of Wnt signaling, results in increased expressions of mDA neuron markers (including A9 subtype mDA neuron markers (e.g., ALDH1A1) and A10 subtype mDA neuron markers (e.g., CALB1) and mDA neuron maturity markers (including, but not limited to, DAT, VMAT2, PITX3, CHRNA6, and CHRNB3). The inhibitor of Wnt signaling can impact the expression of an A9 subtype mDA neuron marker. Non-limiting examples of markers indicating an A9 subtype midbrain dopamine neuron include LMO3, ALDH1A1, SOX6, VGLUT2, and NDNF. In certain embodiments, the inhibitor of Wnt signaling increases the expression of an A9 subtype mDA neuron marker. In certain embodiments, the inhibitor of Wnt signaling increases the expression of ALDH1A1. In certain embodiments, the inhibitor of Wnt signaling increases the expression of an A10 subtype mDA neuron marker. In certain embodiments, the inhibitor of Wnt signaling increases the expression of CALB1.

Furthermore, mDA neurons or precursors thereof generated by the methods disclosed herein have improved fiber outgrowth, reduced remaining Ki67⁺ proliferating cells, and improved in vivo survival, which make these cells more suitable for therapeutic uses. In certain embodiments, the mDA neurons or precursors thereof generated by the methods disclosed herein can survive at least about 2 weeks, at least about 3 weeks, at least about 1 month, at least about 2 months, at least about 3 months, at least about 4 months, at least about 5 months, up to about 6 months, up to about 1 year, up to about 2 years, up to about 3 years, up to about 4 years, or up to about 5 years post in vivo transplantation. In certain embodiments, the mDA neurons generated by the methods disclosed herein can survive up to about 1 month, up to about 2 months, up to about 3 months, up to about 4 months, up to about 5 months, up to about 6 months, up to about 1 year, up to about 2 years, up to about 3 years, up to about 4 years, or up to about 5 years post in vivo transplantation.

### 5.2.1. Stem Cells

The presently disclosed subject matter provides in vitro methods for inducing differentiation of stem cells to produce mDA neurons and precursors thereof. In certain embodiments, the stem cells are pluripotent stem cells. In certain embodiments, the pluripotent stem cells are selected from embryonic stem cells (ESCs), induced pluripotent stem cells (iPSCs), and combinations thereof. In certain embodiments, the stem cells are multipotent stem cells. Non-limiting examples of stem cells that can be used with the presently disclosed methods include nonembryonic stem cells, embryonic stem cells, induced nonembryonic pluripotent cells, and engineered pluripotent cells. In certain embodiments, the stem cells are human stem cells. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell is a human embryonic stem cell (hESC). In certain embodiments, the stem cell is a human induced pluripotent stem cell (hiPSC). In certain embodiments, the stem cells are non-human stem cells. In certain embodiments, the stem cell is a nonhuman primate stem cell. In certain embodiments, the stem cell is a rodent stem cell.

In certain embodiments, the stem cell or a progeny cell thereof contains an introduced heterologous nucleic acid, where said nucleic acid may encode a desired nucleic acid or protein product or have informational value (see, for example, U.S. Pat. No. 6,312,911, which is incorporated by reference in its entirety). Non-limiting examples of protein products include markers detectable via in vivo imaging studies, for example receptors or other cell membrane proteins. Non-limiting examples of markers include fluorescent proteins (such as green fluorescent protein (GFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet, EYFP)), β-galactosidase (LacZ), chloramphenicol acetyltransferase (cat), neomycin phosphotransferase (neo), enzymes (such as oxidases and peroxidases), and antigenic molecules. As used herein, the terms “reporter gene” or “reporter construct” refer to genetic constructs comprising a nucleic acid encoding a protein that is easily detectable or easily assayable, such as a colored protein, fluorescent protein such as GFP or an enzyme such as beta-galactosidase (lacZ gene). In certain embodiments, the reporter can be driven by a recombinant promoter of a premature post-mitotic mDA neuron marker gene, for example, NURR1.

### 5.2.2. SMAD Inhibitors

Non-limiting examples of SMAD inhibitors include inhibitors of transforming growth factor beta (TGFβ)/Activin-Nodal signaling (referred to as “TGFβ/Activin-Nodal inhibitor”), and inhibitors of bone morphogenetic proteins (BMP) signaling. In certain embodiments, the TGFβ/Activin-Nodal inhibitor can neutralize the ligands including TGFβs, BMPs, Nodal, and activins, and/or block their signal pathways through blocking the receptors and downstream effectors. Non-limiting examples of TGFβ/Activin-Nodal inhibitors include those disclosed in WO/2010/096496, WO/2011/149762, WO/2013/067362, WO/2014/176606, WO/2015/077648, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20 (2012), all of which are incorporated by reference in their entireties herein for all purposes. In certain embodiments, the at least one TGFβ/Activin-Nodal inhibitor is selected from inhibitors of ALK5, inhibitors of ALK4, inhibitors of ALK7, and combinations thereof). In certain embodiments, the TGFβ/Activin-Nodal inhibitor comprises an inhibitor of ALK5. In certain embodiments, the TGFβ/Activin-Nodal inhibitor is a small molecule selected from SB431542, derivatives thereof, and mixtures thereof. “SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

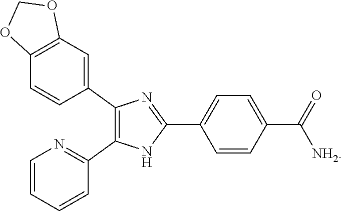 [SMILES: NC(=O)c1ccc(-c2nc(-c3ccc4c(c3)OCO4)c(-c3ccccn3)[nH]2)cc1]

In certain embodiments, the TGFβ/Activin-Nodal inhibitor comprises SB431542. In certain embodiments, the TGFβ/Activin-Nodal inhibitor comprises a derivative of SB431542. In certain embodiments, the derivative of SB431542 is A83-01.

In certain embodiments, the at least one SMAD inhibitor comprises an inhibitor of BMP signaling (referred to as “BMP inhibitor”). Non-limiting examples of BMP inhibitors include those disclosed in WO2011/149762, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20, all of which are incorporated by reference in their entireties. In certain embodiments, the BMP inhibitor is a small molecule selected from LDN193189, Noggin, dorsomorphin, derivatives thereof, and mixtures thereof. “LDN193189” refers to a small molecule DM-3189, IUPAC name 4-(6-(4-(piperazin-1-yl)phenyl)pyrazolo[1,5-a]pyrimidin-3-yl)quinoline, with a chemical formula of C₂₅H₂₂N₆ with the following formula.

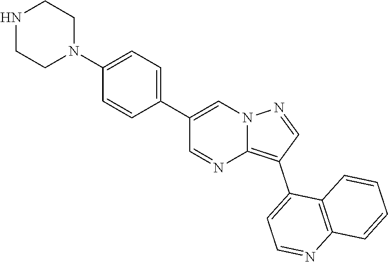 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18: 4388-4392, herein incorporated by reference).

In certain embodiments, the BMP inhibitor comprises LDN193189. In certain embodiments, the BMP inhibitor comprises Noggin.

In certain embodiments, the stem cells are exposed to one SMAD inhibitor, e.g., one TGFβ/Activin-Nodal inhibitor. In certain embodiments, the TGFβ/Activin-Nodal inhibitor is SB431542. In certain embodiments, the TGFβ/Activin-Nodal inhibitor is a derivative of SB431542. In certain embodiments, the TGFβ/Activin-Nodal inhibitor is A83-01.

In certain embodiments, the stem cells are exposed to two SMAD inhibitors. In certain embodiments, the two SMAD inhibitors are a TGFβ/Activin-Nodal inhibitor and a BMP inhibitor. In certain embodiments, the stem cells are exposed to SB431542 or A83-01, and LDN193189 or Noggin. In certain embodiments, the stem cells are exposed to SB431542 and LDN193189. In certain embodiments, the stem cells are exposed to A83-01 and LDN193189. In certain embodiments, the stem cells are exposed to SB431542 and Noggin. In certain embodiments, the stem cells are exposed to A83-01 and Noggin.

In certain embodiments, the stem cells are exposed to or contacted with at least one SMAD inhibitor for at least about 5 days, or at least about 10 days. In certain embodiments, the stem cells are contacted with or exposed to the at least one SMAD inhibitor for up to about 5 days, or up to about 10 days. In certain embodiments, the stem cells are contacted with or exposed to the at least one SMAD inhibitor for between about 5 days and about 10 days. In certain embodiments, the stem cells are contacted with or exposed to the at least one SMAD inhibitor for about 5 days. In certain embodiments, the stem cells are contacted with or exposed to the at least one SMAD inhibitor for 6 days. In certain embodiments, the stem cells are contacted with or exposed to the at least one SMAD inhibitor for 7 days. In certain embodiments, the cells are contacted with or exposed to the at least one SMAD inhibitor from day 0 through day 6. In certain embodiments, the at least one SMAD inhibitor is added every day or every other day to a cell culture medium comprising the stem cells from day 0 through day 6. In certain embodiments, the at least one SMAD inhibitor is added every day (daily) to a cell culture medium comprising the stem cells from day 0 to day 6.

In certain embodiments, the cells are contacted with or exposed to a TGFβ/Activin-Nodal inhibitor. In certain embodiments, the concentration of the TGFβ/Activin-Nodal inhibitor contacted with or exposed to the cells is between about 1 μM and about 20 μM, between about 1 μM and about 10 μM, between about 1 μM and about 15 μM, between about 10 μM and about 15 AM, between about 5 μM and about 10 μM, between about 5 μM and about 15 μM, between about 5 μM and about 20 μM, or between about 15 μM and about 20 μM. In certain embodiments, the concentration of the TGFβ/Activin-Nodal inhibitor contacted with or exposed to the cells is between about 1 μM and about 10 μM. In certain embodiments, the concentration of the TGFβ/Activin-Nodal inhibitor contacted with or exposed to the cells is about 5 μM. about 10 μM. In certain embodiments, the concentration of the TGFβ/Activin-Nodal inhibitor contacted with or exposed to the cells is about 10 μM. In certain embodiments, the TGFβ/Activin-Nodal inhibitor comprises SB431542 or a derivative thereof (e.g., A83-01). In certain embodiments, the TGFβ/Activin-Nodal inhibitor comprises SB431542.

In certain embodiments, the cells are contacted with or exposed to a BMP inhibitor. In certain embodiments, the concentration of the BMP inhibitor contacted with or exposed to the cells is between about 50 nM and about 500 nM, or between about 100 nM and about 500 nM, or between about 200 nM and about 500 nM, or between about 200 and about 300 nM, or between about 200 nM and about 400 nM, or between about 100 nM and about 250 nM, or between about 100 nM and about 250 nM, or between about 200 nM and about 250 nM, or between about 250 nM and about 300 nM. In certain embodiments, the concentration of the BMP inhibitor contacted with or exposed to the cells is between about 200 nM and about 300 mM. In certain embodiments, the concentration of the BMP inhibitor contacted with or exposed to the cells is about 150 nM, about 200 nM, about 250 nM, about 300 nM, or about 350 nM. In certain embodiments, the concentration of the BMP inhibitor contacted with or exposed to the cells is about 250 nM. In certain embodiments, the BMP inhibitor comprises LDN193189 or a derivative thereof. In certain embodiments, the BMP inhibitor comprises LDN193189.

In certain embodiments, the cells are contacted with or exposed to the TGFβ/Activin-Nodal inhibitor and the BMP inhibitor simultaneously. In certain embodiments, the stem cells are contacted with or exposed to the TGFβ/Activin-Nodal inhibitor and the BMP inhibitor for about 5 days. In certain embodiments, the stem cells are contacted with or exposed to the TGFβ/Activin-Nodal inhibitor and the BMP inhibitor for 6 days. In certain embodiments, the stem cells are contacted with or exposed to the TGFβ/Activin-Nodal inhibitor and the BMP inhibitor for 7 days.

In certain embodiments, the cells are contacted with or exposed to the TGFβ/Activin-Nodal inhibitor and the BMP inhibitor from day 0 through day 6. In certain embodiments, the TGFβ/Activin-Nodal inhibitor and the BMP inhibitor are added every day or every other day to a cell culture medium comprising the stem cells from day 0 through day 6. In certain embodiments, the TGFβ/Activin-Nodal inhibitor and the BMP inhibitor are added every day (daily) to a cell culture medium comprising the stem cells from day 0 to day 6.

### 5.2.3. Wnt Activators

In certain embodiments, the at least one Wnt activator lowers GSK3β for activation of Wnt signaling. Thus, in certain embodiments, the Wnt activator is a GSK3β inhibitor. A GSK3β inhibitor is capable of activating a WNT signaling pathway, see e.g., Cadigan, et al., J Cell Sci. 2006; 119:395-402; Kikuchi, et al., Cell Signaling. 2007; 19:659-671, which are incorporated by reference herein in their entireties. As used herein, the term “glycogen synthase kinase 3β inhibitor” or “GSK3β inhibitor” refers to a compound that inhibits a glycogen synthase kinase 3β enzyme, for example, see Doble, et al., J Cell Sci. 2003; 116:1175-1186, which is incorporated by reference herein in its entirety. Non-limiting examples of GSK3β inhibitors include CHIR99021, BIO ((3E)-6-bromo-3-[3-(hydroxyamino)indol-2-ylidene]-1H-indol-2-one), AMBMP hydrochloride, LP 922056, SB-216763, CHIR98014, Lithium, 3F8, deoxycholic acid, and those disclosed in WO2011/149762, WO13/067362, Chambers et al., Nat Biotechnol. 2012 Jul. 1; 30(7):715-20, Kriks et al., Nature. 2011 Nov. 6; 480(7378):547-51, and Calder et al., J Neurosci. 2015 Aug. 19; 35(33):11462-81, all of which are incorporated by reference in their entireties.

Non-limiting examples of Wnt activators include CHIR99021, Wnt3A, Wnt1, Wnt5a, BIO ((3E)-6-bromo-3-[3-(hydroxyamino)indol-2-ylidene]-1H-indol-2-one), AMBMP hydrochloride, LP 922056, SB-216763, CHIR98014, Lithium, 3F8, deoxycholic acid, and those disclosed in WO2011/149762, WO13/067362, Chambers et al., Nat Biotechnol. 2012 Jul. 1; 30(7):715-20, Kriks et al., Nature. 2011 Nov. 6; 480(7378):547-51, and Calder et al., J Neurosci. 2015 Aug. 19; 35(33):11462-81, all of which are incorporated by reference in their entireties. In certain embodiments, the at least one Wnt activator is a small molecule selected from CHIR99021, Wnt3A, Wnt1, Wnt5a, BIO, CHIR98014, Lithium, 3F8, deoxycholic acid, derivatives thereof, and mixtures thereof. In certain embodiments, the at least one Wnt activator comprises CHIR99021 or a derivative thereof. In certain embodiments, the at least one Wnt activator comprises CHIR99021. “CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl)pyrimidin-2-ylamino) ethylamino)nicotinonitrile with the following formula.

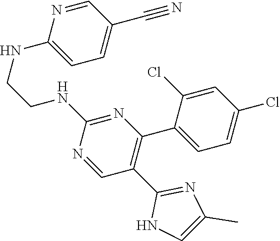 [SMILES: Cc1c[nH]c(-c2cnc(NCCNc3ccc(C#N)cn3)nc2-c2ccc(Cl)cc2Cl)n1]

CHIR99021 is highly selective, showing nearly thousand-fold selectivity against a panel of related and unrelated kinases, with an IC50=6.7 nM against human GSK3β and nanomolar IC50 values against rodent GSK3β homologs.

In certain embodiments, the cells are contacted with or exposed to the at least one Wnt activator for at least about 5 days, at least about 10 days, at least about 15 days, or at least about 20 days. In certain embodiments, the cells are contacted with or exposed to the at least one Wnt activator for up to about 5 days, up to about 10 days, up to about 15 days, or up to about 20 days. In certain embodiments, the cells are contacted with or exposed to the at least one Wnt activator for between about 5 days and about 20 days, between about 5 days and about 15 days, between about 10 days and about 20 days, between about 5 days and about 15 days, or between about 10 days and about 15 days. In certain embodiments, the cells are contacted with the at least one Wnt activator for between about 10 days and about 20 days. In certain embodiments, the cells are contacted with the at least one Wnt activator for about 15 days. In certain embodiments, the stem cells are contacted with the at least one activator of Wnt signaling for 16 days. In certain embodiments, the stem cells are contacted with the at least one activator of Wnt signaling for 17 days. In certain embodiments, the cells are contacted with the at least one Wnt activator from day 0 through day 16. In certain embodiments, the at least one Wnt activator is added every day or every other day to a cell culture medium comprising the cells from day 0 through day 16. In certain embodiments, the at least one Wnt activator is added every day (daily) to a cell culture medium comprising the cells from day 0 through day 16.

In certain embodiments, the concentration of the at least Wnt activator is increased during its exposure to the cells (also referred to as “Wnt Boost”). In certain embodiments, the increase or Wnt Boost is initiated at least about 2 days, at least about 4 days, or at least about 5 days from the initial exposure of the cells to the at least one Wnt activator. In certain embodiments, the increase or Wnt Boost is initiated about 4 days from the initial exposure of the cells to the at least one Wnt activator.

In certain embodiments, the cells are contacted with or exposed to the increased concentration of the at least one Wnt activator for at least about 5 days, or at least about 10 days. In certain embodiments, the cells are contacted with or exposed to the increased concentration of the at least one Wnt activator for at least about 5 days. In certain embodiments, the cells are contacted with the increased concentration of the at least one Wnt activator for up to about 5 days, up to about 10 days, or up to about 15 days. In certain embodiments, the cells are contacted with the increased concentration of the at least one Wnt activator for up to about 10 days.

In certain embodiments, the cells are contacted with or exposed to the increased concentration of the at least one Wnt activator for between about 5 days and about 15 days, or between about 5 days and about 10 days, or between about 10 days and about 15 days. In certain embodiments, the cells are contacted with or exposed to the increased concentration of the at least one Wnt activator for between about 5 days and about 10 days. In certain embodiments, the cells are contacted with or exposed to the increased concentration of the at least one Wnt activator for about 5 days, about 10 days, or about 15 days. In certain embodiments, the cells are contacted with or exposed to the increased concentration of the at least one Wnt activator for about 5 days. In certain embodiments, the cells are contacted with or exposed to the increased concentration of the at least one Wnt activator for 5 days. In certain embodiments, the cells are contacted with or exposed to the increased concentration of the at least one Wnt activator for 6 days. In certain embodiments, the cells are contacted with or exposed to the increased concentration of the at least one Wnt activator from day 4 through day 9. In certain embodiments, the cells are contacted with or exposed to the increased concentration of the at least one Wnt activator for about 10 days. In certain embodiments, the cells are contacted with or exposed to the increased concentration of the at least one Wnt activator for 12 days. In certain embodiments, the cells are contacted with or exposed to the increased concentration of the at least one Wnt activator for 13 days. In certain embodiments, the cells are contacted with or exposed to the increased concentration of the at least one Wnt activator from day 4 through day 16.

In certain embodiments, the initial concentration of the at least one Wnt activator contacted with or exposed to the cells prior to the Wnt Boost is less than about 5 M, less than about 3 μM, or less than about 1.5 μM, or less than about 1.5 μM, including, but not limited to, between about 0.01 μM and about 5 μM, between about 0.01 μM and about 3 μM, between about 0.05 μM and about 3 μM, between about 0.1 μM and about 3 μM, between about 0.5 μM and about 3 μM, between about 0.5 μM and about 2 μM, between about 0.5 μM and about 1 μM, or between about 0.5 μM and about 1.5 μM. In certain embodiments, the initial concentration of the at least one Wnt activator contacted with or exposed to the cells prior to the Wnt Boost is about 1 μM. In certain embodiments, the initial concentration of the at least one Wnt activator contacted with or exposed to the cells prior to the Wnt Boost is less than about 1.5 μM, e.g., about 1 μM, about 0.1 M, about 0.2 μM, about 0.3 μM, about 0.4 μM, about 0.5 μM, about 0.6 μM, about 0.7 μM, about 0.8 M, or about 0.9 μM. In certain embodiments, the initial concentration of the at least one Wnt activator contacted with or exposed to the cells prior to the Wnt boost is about 1 μM. In certain embodiments, the initial concentration of the at least one Wnt activator contacted with or exposed to the cells prior to the Wnt boost is about 0.5 μM. In certain embodiments, the initial concentration of the at least one Wnt activator contacted with or exposed to the cells prior to the Wnt boost is about 0.7 μM.

In certain embodiments, the increased concentration of the at least one Wnt activator post the Wnt Boost is about 3 μM or greater, about 5 μM or greater, about 10 μM or greater, about 15 μM or greater, or about 20 μM or greater. In certain embodiments, the increased concentration of the at least one Wnt activator post the Wnt Boost is between about 3 μM and about 15 μM, between about 3 μM and about 10 μM, or between about 5 μM and about 10 μM. In certain embodiments, the increased concentration of the at least one Wnt activator post the Wnt Boost is between about 5 μM and about 10 μM. In certain embodiments, the increased concentration of the at least one Wnt activator post the Wnt Boost is about 3 μM, about 3.5 μM, about 4 μM, about 4.5 μM, about 5 μM, about 5.5 μM, about 6 μM, about 6.5 μM, about 7 μM, about 7.5 μM, about 8 μM, about 8.5 M, about 9 μM, about 9.5 μM, or about 10 μM. In certain embodiments, the increased concentration of the at least one Wnt activator post the Wnt Boost is about 3 μM. In certain embodiments, the increased concentration of the at least one Wnt activator post the Wnt boost is about 6 μM. In certain embodiments, the increased concentration of the at least one Wnt activator post the Wnt boost is about 7 μM. In certain embodiments, the increased concentration of the at least one Wnt activator post the Wnt Boost is about 7.5 μM.

In certain embodiments, the concentration of the at least one Wnt activator is increased from the initial concentration contacted with or exposed to the cells by between about 50% and about 2000%, or between about 100% and about 1500%, or between about 150% and about 1500%, or between about 200% and about 1500%, or between about 250% and about 1500%, or between about 300% and about 1500%, or between about 300% and about 1000%, or between about 300% and about 400%, or between about 500% and about 1000%, or between about 800% and about 1000%, or between about 900% and about 1000%, or between about 950% and about 1000%. In certain embodiments, the concentration of the at least one Wnt activator is increased from the initial concentration contacted with or exposed to the cells by between about 300% and about 1000%. In certain embodiments, the concentration of the at least one Wnt activator is increased from the initial concentration contacted with or exposed to the cells by between about 300% and about 500%. In certain embodiments, the concentration of the at least one Wnt activator is increased from the initial concentration contacted with or exposed to the cells by between about 900% and about 1000%. In certain embodiments, the concentration of the at least one Wnt activator is increased from the initial concentration contacted with or exposed to the cells by about 200%, about 250%, about 300%, about 350%, about 400%, about 450%, about 500%, about 550%, about 600%, about 650%, about 700%, about 750%, about 800%, about 850%, about 900%, about 950%, about 1000%, about 1050%, or about 1100%. In certain embodiments, the concentration of the at least one Wnt activator is increased from the initial concentration contacted with or exposed to the cells by about 200%. In certain embodiments, the concentration of the at least one Wnt activator is increased from the initial concentration contacted with or exposed to the cells by about 300%. In certain embodiments, the concentration of the at least one Wnt activator is increased from the initial concentration contacted with or exposed to the cells by about 350%. In certain embodiments, the concentration of the at least one Wnt activator is increased from the initial concentration contacted with or exposed to the cells by about 500%. In certain embodiments, the concentration of the at least one Wnt activator is increased from the initial concentration contacted with or exposed to the cells by about 950%. In certain embodiments, the concentration of the at least one Wnt activator is increased from the initial concentration contacted with or exposed to the cells by about 1000%.

In certain embodiments, the concentration of the at least one Wnt activator is increased from about 1 μM to between about 5 μM and about 10 μM. In certain embodiments, the concentration of the at least one Wnt activator is increased from about 1 μM to about 6 μM. In certain embodiments, the concentration of the at least one Wnt activator is increased from about 1 μM to between about 3 μM and about 5 μM. In certain embodiments, the concentration of the at least one Wnt activator is increased from about 1 μM to about 3 μM.

In certain embodiments, the at least one Wnt activator comprises a GSK3β inhibitor. In certain embodiments, the at least one Wnt activator comprises CHIR99021 or a derivative thereof. In certain embodiments, the at least one Wnt activator comprises CHIR99021.

### 5.2.4. SHH Activators

As used herein, the term “Sonic hedgehog,” “SHH,” or “Shh” refers to a protein that is one of at least three proteins in the mammalian signaling pathway family called hedgehog, another is desert hedgehog (DHH) wile a third is Indian hedgehog (IHH). SHH interacts with at least two transmembrane proteins by interacting with transmembrane molecules Patched (PTC) and Smoothened (SMO). SHH typically binds to PTC, which then allows the activation of SMO as a signal transducer. In the absence of SHH, PTC typically inhibits SMO, which in turn activates a transcriptional repressor so transcription of certain genes does not occur. When SHH is present and binds to PTC, PTC cannot interfere with the functioning of SMO. With SMO uninhibited, certain proteins are able to enter the nucleus and act as transcription factors allowing certain genes to be activated (see Gilbert, 2000 Developmental Biology (Sunderland, Mass., Sinauer Associates, Inc., Publishers). In certain embodiments, an SHH activator refers to any molecule or compound that is capable of activating a SHH signaling pathway, including a molecule or compound that is capable of binding to PTC or a SMO. In certain embodiments, the at least one SHH activator is selected from the group consisting of molecules that bind to PCT, molecules that bind to SMO, and combinations thereof. Non-limiting examples of SHH activators include those disclosed in WO10/096496, WO13/067362, Chambers et al., Nat Biotechnol. 2009 March; 27(3):275-80, and Kriks et al., Nature. 2011 Nov. 6; 480(7378):547-51. In certain embodiments, the at least one SHH activator is selected from the group consisting of a SHH protein, a SMO agonist, or a combination thereof. In certain embodiments, the SHH protein is selected from the group consisting of a recombinant SHH, a modified N-terminal SHH, or a combination thereof. In certain embodiments, the recombinant SHH comprises a N-terminal fragment and a C-terminal fragment. In certain embodiments, the modified N-terminal SHH comprises two Isoleucines at the N-terminus. In certain embodiments, the modified N-terminal SHH has at least about 80%, about 85%, about 90%, about 95%, or about 99% sequence identity to an un-modified N-terminal SHH. In certain embodiments, the modified N-terminal SHH has at least about 80%, about 85%, about 90%, about 95%, or about 99% sequence identity to an un-modified human N-terminal SHH. In certain embodiments, the modified N-terminal SHH has at least about 80%, about 85%, about 90%, about 95%, or about 99% sequence identity to an un-modified mouse N-terminal SHH. In certain embodiments, the modified N-terminal SHH comprises SHH C25II. In certain embodiments, the modified N-terminal SHH comprises SHH C24II.

Non-limiting examples of SMO agonists (SAGs) include purmorphamine, GSA10, and 20(S)-hydroxy Cholesterol. In certain embodiments, the SAG comprises purmorphamine.

In certain embodiments, the cells are contacted with or exposed to the at least one SHH activator for at least about 5 days, or at least about 10 days. In certain embodiments, the cells are contacted with or exposed to the at least one SHH activator for up to about 5 days, or up to about 10 days. In certain embodiments, the cells are contacted with or exposed to the at least one SHH activator for between about 5 days and about 10 days. In certain embodiments, the cells are contacted with or exposed to the at least one SHH activator for about 5 days. In certain embodiments, the cells are contacted with or exposed to the at least one SHH activator for 6 days. In certain embodiments, the cells are contacted with or exposed to the at least one SHH activator for 7 days. In certain embodiments, the cells are contacted with or exposed to the at least one SHH activator from day 0 through day 6. In certain embodiments, the at least one SHH activator is added every day or every other day to a cell culture medium comprising the cells from day 0 through day 6. In certain embodiments, the at least one SHH activator is added every day (daily) to a cell culture medium comprising the cells from day 0 through day 6.

In certain embodiments, the concentration of the at least one SHH activator contacted with or exposed to the cells is between about 50 ng/mL and about 1000 ng/mL, between about 100 ng/mL and about 1000 ng/mL, between about 20 ng/mL and about 1000 ng/mL, between about 300 ng/mL and about 1000 ng/mL, between about 400 ng/mL and about 1000 ng/mL, between about 500 ng/mL and about 1000 ng/mL, between about 400 ng/mL and about 800 ng/mL, between about 400 ng/mL and about 700 ng/mL, between about 400 ng/mL and about 600 ng/mL, or between about 500 ng/mL and about 600 ng/mL. In certain embodiments, the concentration of the at least one SHH activator contacted with or exposed to the cells is between about 400 ng/mL and about 600 ng/mL. In certain embodiments, the concentration of the at least one SHH activator contacted with or exposed to the cells is about 400 ng/mL, about 450 ng/mL, about 500 ng/mL, about 550 ng/mL, or about 600 ng/mL. In certain embodiments, the concentration of the at least one SHH activator contacted with or exposed to the cells is about 500 ng/mL.

In certain embodiments, the at least one activator of SHH signaling comprises SHH C25II.

### 5.2.5. FGF Activators

FGF family includes secreted signaling proteins (secreted FGFs) that signal to receptor tyrosine kinases. Phylogenetic analysis suggests that 22 Fgf genes can be arranged into seven subfamilies containing two to four members each. Branch lengths are proportional to the evolutionary distance between each gene.

In certain embodiments, the at least one FGF activator is selected from the group consisting of FGF8a, FGF17, FGF18, FGF8b, FGF2, FGF4, and derivatives thereof. In certain embodiments, the at least one FGF activator is selected from the group consisting of FGF8a, FGF17, FGF18, FGF2, FGF4, and derivatives thereof. In certain embodiments, the at least one FGF activator is selected from the group consisting of FGF8a, FGF17, and FGF18.

The FGF8 subfamily is comprised of FGF8a, FGF8b, FGF17, and FGF18. Early patterning of the vertebrate midbrain and cerebellum is regulated by a mid/hindbrain organizer that produces FGF8a, FGF8b, FGF17 and FGF18. It has been shown that FGF8b functions differently from FGF8a, FGF17, and FGF18 (Liu et al., Development. 2003 December; 130(25):6175-85). FGF8b is the only protein that can induce the r1 gene Gbx2 and strongly activate the pathway inhibitors Spry1/2, as well as repress the midbrain gene Otx2 (Liu 2003). Moreover, FGF8b extends the organizer along the junction between the induced Gbx2 domain and the remaining Otx2 region in the midbrain, correlating with cerebellum development (Liu 2003). By contrast, FGF8a, FGF17, and FGF18 cause expansion of the midbrain and upregulating midbrain gene expression (Liu 2003).

In certain embodiments, the at least one FGF activator is capable of causing expansion of the midbrain and upregulating midbrain gene expression. In certain embodiments, the at least one FGF activator is capable of promoting midbrain development. In certain embodiments, the at least one FGF activator is selected from the group consisting of FGF8a, FGF17, FGF18, FGF2, FGF4, derivatives thereof, and combinations thereof. In certain embodiments, the at least one FGF activator is selected from the group consisting of FGF8a, FGF17, FGF18, and combinations thereof. In certain embodiments, the at least one FGF activator comprises or is FGF18.

In certain embodiments, the cells are contacted with or exposed to the at least one FGF activator for at least about 1 day, at least about 3 days, at least about 5 days, at least about 8 days, or at least about 10 days. In certain embodiments, the cells are contacted with or exposed to the at least one FGF activator for at least about 5 days. In certain embodiments, the cells are contacted with or exposed to the at least one FGF activator for at least 4 days. In certain embodiments, the cells are contacted with or exposed to the at least one FGF activator for up to about 5 days (e.g., up to 5 days, up to 6 days, or up to 7 days), or up to about 10 days (e.g., up to 8 days, up to 9 days, up to 10 days, up to 11 days, up to 12 days), or up to about 15 days, or up to about 20 days. In certain embodiments, the cells are contacted with or exposed to the at least one FGF activator for at least 4 days and/or for up to 7 days. In certain embodiments, the cells are contacted with or exposed to the at least one FGF activator for between about 1 days and about 20 days, between about 1 day and about 15 days, between about 1 day and about 5 days, between about 5 days and about 20 days, between about 5 days and about 15 days, or between about 5 days and about 10 days, between about 10 days and about 20 days. In certain embodiments, the cells are contacted with or exposed to the at least one FGF activator for between about 1 day and about 10 days. In certain embodiments, the cells are contacted with or exposed to the at least one FGF activator for about 3 days, about 5 days, or about 8 days. In certain embodiments, the cells are contacted with or exposed to the at least one FGF activator for between about 1 days and about 5 days. In certain embodiments, the cells are contacted with or exposed to the at least one FGF activator for about 5 days. In certain embodiments, the cells are contacted with or exposed to the at least one FGF activator for about 4 days. In certain embodiments, the cells are contacted with or exposed to the at least one FGF activator for 5 days.

In certain embodiments, the contact of the cells with or the exposure of the cells to the at least one FGF activator is initiated at least about 5 days, or at least about 10 days from the initial contact of the cells with or the initial exposure of the cells to the at least one SMAD inhibitor. In certain embodiments, the contact of the cells with or the exposure of the cells to the at least one FGF activator is initiated no later than about 15 days or no later than about 20 days from the initial contact of the cells with or the initial exposure of the cells to the at least one SMAD inhibitor. In certain embodiments, the contact of the cells with or the exposure of the cells to the at least one FGF activator is initiated no later than 18 days from the initial contact of the cells with or the initial exposure of the cells to the at least one SMAD inhibitor. In certain embodiments, the contact of the cells with or the exposure of the cells to the at least one FGF activator is initiated between about 5 days and about 20 days, between about 5 days and about 20 days, between about 10 days and about 15 days, between about 10 days and 18 days, between about 5 days and about 15 days, or between about 10 days and about 20 days, from the initial contact of the cells with or the initial exposure of the cells to the at least one SMAD inhibitor. In certain embodiments, the contact of the cells with or the exposure of the cells to the at least one FGF activator is initiated between about 5 days and about 10 days from the initial contact of the cells with or the initial exposure of the cells to the at least one SMAD inhibitor. In certain embodiments, the contact of the cells with or the exposure of the cells to the at least one FGF activator is initiated about 10 days from the initial contact of the cells with or the initial exposure of the cells to the at least one SMAD inhibitor. In certain embodiments, the contact of the cells with or the exposure of the cells to the at least one FGF activator is initiated 12 days from the initial contact of the cells with or the initial exposure of the cells to the at least one SMAD inhibitor. In certain embodiments, the contact of the cells with or the exposure of the cells to the at least one FGF activator is initiated 13 days from the initial contact of the cells with or the initial exposure of the cells to the at least one SMAD inhibitor.

In certain embodiments, the contact of the cells with or the exposure of the cells to the at least one FGF activator is initiated about 10 days from the initial contact of the cells with or the initial exposure of the cells to the at least one SMAD inhibitor, and the cells are contacted with the at least FGF activator for about 5 days. In certain embodiments, the contact of the cells with or the exposure of the cells to the at least one FGF activator is initiated 12 days or 13 days from the initial contact of the cells with or the initial exposure of the cells to the at least one SMAD inhibitor, and the cells are contacted with the at least one FGF activator for 4 days or 5 days. In certain embodiments, the cells are contacted with or exposed to the at least one FGF activator from day 12 through day 16. In certain embodiments, the at least one FGF activator is added every day or every other day to a cell culture medium comprising the cells from day 12 through day 16. In certain embodiments, the at least one FGF activator is added every day (daily) to a cell culture medium comprising the cells from day 12 through day 16.

In certain embodiments, the concentration of the at least one FGF activator contacted with or exposed to the cells is between about 10 ng/mL and about 500 ng/mL, between about 50 ng/mL and about 500 ng/mL, between about 100 ng/mL and about 500 ng/mL, between about 100 ng/mL and about 400 ng/mL, between about 100 ng/mL and about 300 ng/mL, between about 100 ng/mL and about 200 ng/mL, or between about 100 ng/mL and about 250 ng/mL. In certain embodiments, the concentration of the at least one FGF activator contacted with or exposed to the cells is between about 100 ng/mL and about 200 ng/mL. In certain embodiments, the concentration of the at least one FGF activator contacted with or exposed to the cells is about 100 ng/mL. In certain embodiments, concentration of the at least one FGF activator contacted with or exposed to the cells is about 200 ng/mL.

In certain embodiments, the at least one FGF activator comprises FGF18.

### 5.2.6. Wnt Inhibitors

Wnt signaling includes canonical Wnt signaling and non-canonical Wnt signaling. In certain embodiments, the at least one Wnt inhibitor is capable of inhibiting canonical Wnt signaling. In certain embodiments, the at least one Wnt inhibitor is capable of inhibiting both canonical Wnt signaling and non-canonical Wnt signaling. Non-limiting examples of Wnt inhibitors that are capable of inhibiting both canonical Wnt signaling and non-canonical Wnt signaling include IWP2, IWR1-endo, IWP-O1, Wnt-C59, IWP-L6, IWP12, LGK-974, IWR-1, ETC-159, iCRT3, IWP-4, Salinomycin, Pyrvinium Pamoate, iCRT14, FH535, CCT251545, Wogonin, NCB-0846, Hexachrorophene, KY02111, S03031 (KY01-I), S02031 (KY02-I), BC2059, PKF115-584, Quercetin, NSC668036, G007-LK, and derivatives thereof. In certain embodiments, the at least one Wnt inhibitor is selected from the group consisting of IWP2, IWR1-endo, XAV939, IWP-O1, Wnt-C59, IWP-L6, LGK-974, IWR-1, Wnt-C59, ETC-159, iCRT3, IWP-4, ICG-001, Salinomycin, Pyrvinium Pamoate, iCRT14, FH535, CCT251545, KYA1797K, Wogonin, NCB-0846, Hexachrorophene, PNU-74654, KY02111, S03031 (KY01-I), S02031 (KY02-I), Triptonide, IWP12, BC2059, PKF115-584, Quercetin, NSC668036, G007-LK, MSAB, LF3, JW55, Isoquercitrin, WIKI4 (Wnt Inhibitor Kinase Inhibitor 4), derivatives thereof, and combinations thereof. In certain embodiments, the at least one inhibitor of Wnt signaling is selected from the group consisting of IWP2, IWR1-endo, IWP-O1, IWP12, Wnt-C59, IWP-L6, LGK-974, IWR-1, ETC-159, iCRT3, IWP-4, Salinomycin, Pyrvinium Pamoate, iCRT14, FH535, CCT251545, Wogonin, NCB-0846, Hexachrorophene, KY02111, S03031 (KY01-I), S02031 (KY02-I), BC2059, PKF115-584, Quercetin, NSC668036, G007-LK, derivatives thereof, and combinations thereof. In certain embodiments, the at least one inhibitor of Wnt signaling is selected from the group consisting of XAV939, ICG-001, PNU-74654, Triptonide, KYA1797K, MSAB, LF3, JW55, Isoquercitrin, WIKI4, derivatives thereof, and combinations thereof. In certain embodiments, the at least one Wnt inhibitor comprises IWP2 or a derivative thereof.

In certain embodiments, the cells are contacted with or exposed to the at least one Wnt inhibitor for at least about 1 day, at least about 3 days, at least about 5 days, at least about 8 days, at least about 10 days, at least about 15 days, or at least about 20 days. In certain embodiments, the cells are contacted with or exposed to the at least one Wnt inhibitor for up to about 5 days, or up to about 10 days, or up to about 15 days, up to about 20 days, up to about 25 days, or up to about 30 days. In certain embodiments, the cells are contacted with or exposed to the at least one Wnt inhibitor for between about 1 days and about 20 days, between about 1 day and about 15 days, between about 1 day and about 5 days, between about 5 days and about 20 days, between about 5 days and about 15 days, or between about 5 days and about 10 days, between about 10 days and about 20 days, between about 10 days and about 15 days, or between about 15 days and about 20 days, between about 10 days and about 30 days, between about 10 days and about 25 days, between about 15 days and about 30 days, between about 15 days and about 25 days, between about 20 days and about 30 days, between about 20 days and about 25 days, or between about 25 days and about 30 days. In certain embodiments, the cells are contacted with or exposed to the at least one Wnt inhibitor for between about 1 day and about 10 days. In certain embodiments, the cells are contacted with or exposed to the at least one Wnt inhibitor for between about 10 day and about 15 days. In certain embodiments, the cells are contacted with or exposed to the at least one Wnt inhibitor for between about 15 day and about 20 days. In certain embodiments, the cells are contacted with or exposed to the at least one Wnt inhibitor for about 5 days. In certain embodiments, the cells are contacted with or exposed to the at least one Wnt inhibitor for about 15 days. In certain embodiments, the cells are contacted with or exposed to the at least one Wnt inhibitor for about 20 days. In certain embodiments, the cells are contacted with or exposed to the at least one Wnt inhibitor for 4 days. In certain embodiments, the cells are contacted with or exposed to the at least one Wnt inhibitor for 5 days. In certain embodiments, the cells are contacted with or exposed to the at least one Wnt inhibitor for 6 days. In certain embodiments, the cells are contacted with or exposed to the at least one Wnt inhibitor for 7 days. In certain embodiments, the cells are contacted with or exposed to the at least one Wnt inhibitor for 14 days. In certain embodiments, the cells are contacted with or exposed to the at least one Wnt inhibitor for 15 days. In certain embodiments, the cells are contacted with or exposed to the at least one Wnt inhibitor for 19 days. In certain embodiments, the cells are contacted with or exposed to the at least one Wnt inhibitor for 20 days. In certain embodiments, the cells are contacted with or exposed to the at least one Wnt inhibitor for 16 days, 17 days, 18 days, 21 days, 22 days, or 23 days.

In certain embodiments, the cells that are contacted with the at least one Wnt inhibitor comprise mDA neuron precursors and mDA neurons.

In certain embodiments, the contact of the cells with or the exposure of the cells to the at least one Wnt inhibitor is initiated at least about 5 days, or at least about 10 days from the initial contact of the cells with or the initial exposure of the cells to the at least one SMAD inhibitor. In certain embodiments, the contact of the cells with or the exposure of the cells to the at least one Wnt inhibitor is initiated no later than about 15 days or no later than about 20 days from the initial contact of the cells with or the initial exposure of the cells to the at least one SMAD inhibitor. In certain embodiments, the contact of the cells with or the exposure of the cells to the at least one Wnt inhibitor is initiated between about 5 days and about 20 days, between about 5 days and about 20 days, between about 10 days and about 15 days, between about 5 days and about 15 days, or between about 10 days and about 20 days, from the initial contact of the cells with or the initial exposure of the cells to the at least one SMAD inhibitor. In certain embodiments, the contact of the cells with or the exposure of the cells to the at least one Wnt inhibitor is initiated between about 5 days and about 10 days from the initial contact of the cells with or the initial exposure of the cells to the at least one SMAD inhibitor. In certain embodiments, the contact of the cells with or the exposure of the cells to the at least one Wnt inhibitor is initiated about 10 days from the initial contact of the cells with or the initial exposure of the cells to the at least one SMAD inhibitor. In certain embodiments, the contact of the cells with or the exposure of the cells to the at least one Wnt inhibitor is initiated 10 days from the initial contact of the cells with or the initial exposure of the cells to the at least one SMAD inhibitor. In certain embodiments, the contact of the cells with or the exposure of the cells to the at least one Wnt inhibitor is initiated 11 days from the initial contact of the cells with or the initial exposure of the cells to the at least one SMAD inhibitor. In certain embodiments, the contact of the cells with or the exposure of the cells to the at least one Wnt inhibitor is initiated 12 days from the initial contact of the cells with or the initial exposure of the cells to the at least one SMAD inhibitor. In certain embodiments, the contact of the cells with or the exposure of the cells to the at least one Wnt inhibitor is initiated 13 days from the initial contact of the cells with or the initial exposure of the cells to the at least one SMAD inhibitor.

In certain embodiments, the contact of the cells with or the exposure of the cells to the at least one Wnt inhibitor is initiated about 10 days from the initial contact of the cells with or the initial exposure of the cells to the at least one SMAD inhibitor, and the cells are contacted with the at least Wnt inhibitor for about 5 days. In certain embodiments, the contact of the cells with or the exposure of the cells to the at least one Wnt inhibitor is initiated 12 days or 13 days from the initial contact of the cells with or the initial exposure of the cells to the at least one SMAD inhibitor, and the cells are contacted with the at least one Wnt inhibitor for 4 days or 5 days. In certain embodiments, the cells are contacted with or exposed to the at least one Wnt inhibitor from day 12 through day 16. In certain embodiments, the at least one Wnt inhibitor is added every day or every other day to a cell culture medium comprising the cells from day 12 through day 16. In certain embodiments, the at least one Wnt inhibitor is added every day (daily) to a cell culture medium comprising the cells from day 12 through day 16. In certain embodiments, the cells are contacted with or exposed to the at least one Wnt inhibitor from day 12 through day 25. In certain embodiments, the at least one Wnt inhibitor is added every day or every other day to a cell culture medium comprising the cells from day 12 through day 25. In certain embodiments, the at least one Wnt inhibitor is added every day (daily) to a cell culture medium comprising the cells from day 12 through day 25. In certain embodiments, the cells are contacted with or exposed to the at least one Wnt inhibitor from day 12 through day 30. In certain embodiments, the at least one Wnt inhibitor is added every day or every other day to a cell culture medium comprising the cells from day 12 through day 30. In certain embodiments, the at least one Wnt inhibitor is added every day (daily) to a cell culture medium comprising the cells from day 12 through day 30. In certain embodiments, the cells are contacted with or exposed to the at least one Wnt inhibitor and the at least one FGF activator simultaneously. In certain embodiments, the at least one Wnt inhibitor and the at least one FGF activator together are added to a cell culture medium comprising the cells.

In certain embodiments, the concentration of the at least one Wnt inhibitor contacted with or exposed to the cells is between about 0.5 μM and about 20 μM, between about 0.5 μM and about 10 μM, between about 0.5 μM and about 5 μM, between about 0.5 μM and about 1 M, between about 0.5 μM and about 2 μM, between about 5 μM and about 10 μM, between about 10 μM and about 20 μM, between about 1 μM and about 2 μM, or between about 1 μM and about 5 μM. In certain embodiments, the concentration of the at least one Wnt inhibitor contacted with or exposed to the cells is between about 0.5 μM and about 2 μM. In certain embodiments, the concentration of the at least one Wnt inhibitor contacted with or exposed to the cells is about 1 μM.

In certain embodiments, the at least one Wnt inhibitor comprises IWP2.

### 5.2.7. Exemplary Methods

In certain embodiments, the stem cells are contacted with or exposed to at least one TGFβ/Activin-Nodal inhibitor (e.g., SB431542, e.g., at a concentration of about 10 μM), at least one BMP inhibitor (e.g., LDN193189, e.g., at a concentration of about 250 nM), and at least one SHH activator (e.g., SHH C25II, e.g., a concentration of about 500 ng/mL) for about 5 days (e.g., 7 days, e.g., from day 0 to day 6), and the cells are contacted with the at least one Wnt activator (e.g., CHIR99021, e.g., at a concentration of about 1 μM for about 5 days (e.g., 4 days, e.g., from day 0 to day 3), and at a concentration of about 6 μM for about 5 days (e.g., 6 days, e.g., from day 4 to day 9), and at a concentration of about 3 μM for about 5 days (e.g., 7 days, e.g., from day 10 to day 16). The cells are contacted with or exposed to the at least one FGF activator (e.g., FGF18, e.g., at a concentration of about 100 ng/ml), wherein the contact of the cells with the at least one FGF activator is initiated about 10 days (e.g., 10 days or 12 days) from the initial contact of the cells with the at least one SMAD inhibitor, and the cells are contacted with the at least one FGF activator for about 5 days (e.g., 5 days (from day 12 to day 16) or 7 days (e.g., from day 10 to day 16). The cells are contacted with or exposed to the at least one Wnt inhibitor (e.g., IWP2, e.g., at a concentration of about 1 μM), wherein the contact of the cells with the at least one Wnt inhibitor is initiated about 10 days (e.g., 10 days or 12 days) from the initial contact of the cells with the at least one SMAD inhibitor, and the cells are contacted with the at least one Wnt inhibitor for about 5 days (e.g., 5 days (from day 12 to day 16), 7 days (e.g., from day 10 to day 16), about 15 days (e.g., 14 days (from day 12 to day 25), or about 20 days (e.g., 19 days (from day 12 to day 30).

### 5.2.8. Cell Culture Media

In certain embodiments, the above-described inhibitors and activators are added to a cell culture medium comprising the cells. Suitable cell culture media include, but are not limited to, Knockout® Serum Replacement (“KSR”) medium, Neurobasal® medium (NB), N2 medium, B-27 medium, and Essential 8®/Essential 6® (“E8/E6”) medium, and combinations thereof. KSR medium, NB medium, N2 medium, B-27 medium, and E8/E6 medium are commercially available. KSR medium is a defined, serum-free formulation optimized to grow and maintain undifferentiated hESCs in culture.

In certain embodiments, the cell culture medium is a KSR medium. The components of a KSR medium are disclosed in WO2011/149762. In certain embodiments, a KSR medium comprises Knockout DMEM, Knockout Serum Replacement, L-Glutamine, Pen/Strep, MEM, and 13-mercaptoethanol. In certain embodiments, 1 liter of KSR medium comprises 820 mL of Knockout DMEM, 150 mL of Knockout Serum Replacement, 10 mL of 200 mM L-Glutamine, 10 mL of Pen/Strep, 10 mL of 10 mM MEM, and 55 μM of 13-mercaptoethanol.

In certain embodiments, the cell culture medium is an E8/E6 medium. E8/E6 medium is a feeder-free and xeno-free medium that supports the growth and expansion of human pluripotent stem cells. E8/E6 medium has been proven to support somatic cell reprogramming. In addition, E8/E6 medium can be used as a base for the formulation of custom media for the culture of PSCs. One example E8/E6 medium is described in Chen et al., Nat Methods 2011 May; 8(5):424-9, which is incorporated by reference in its entirety. One example E8/E6 medium is disclosed in WO15/077648, which is incorporated by reference in its entirety. In certain embodiments, an E8/E6 cell culture medium comprises DMEM/F12, ascorbic acid, selenium, insulin, NaHCO₃, transferrin, FGF2 and TGFβ. The E8/E6 medium differs from a KSR medium in that E8/E6 medium does not include an active BMP ingredient. Thus, in certain embodiments, when an E8/E6 medium is used to culture the presently disclosed stem cells to differentiate into mDA neurons or precursors thereof, at least one BMP inhibitor is not required to be added to the E8/E6 medium. In certain embodiments, the when an E8/E6 medium is used to culture the presently disclosed stem cells to differentiate into mDA neurons or precursors thereof, at least one BMP inhibitor is added to the E8/E6 medium.

### 5.2.9. Differentiated Cells

In certain embodiments, the method comprises obtaining a cell population of the differenced cells, wherein at least about 10%, at least about 20%, at least about 30%, at least about 40%, at least about 50%, at least about 60%, at least about 70%, at least about 80%, or at least about 90% of the differentiated cells express at least one marker indicating a mDA neuron or a precursor thereof. Non-limiting examples of markers indicating a mDA neuron or a precursor thereof include engrailed-1 (EN1), orthodenticle homeobox 2 (OTX2), tyrosine hydroxylase (TH), nuclear receptor related-1 protein (NURR1), forkhead box protein A2 (FOXA2), and LIM homeobox transcription factor 1 alpha (LMX1A), PITX3, LMO3, SNCA, ADCAP1, CHRNA4, ALDH1A1, DAT, VMAT1, SOX6, WNT1, and GIRK2.

In certain embodiments, the differentiated cells express the at least one marker indicating a mDA neuron or a precursor thereof at least about 10 days (e.g., about 15 days, about 20 days, about 30 days, about 40 days, or about 50 days) from the initial contact of the cells with the at least one SMAD inhibitor. In certain embodiments, the differentiated cells express the at least one marker indicating a mDA neuron or a precursor thereof about 15 days (e.g., 15 days, 16 days, or 17 days) from the initial contact of the cells with the at least one SMAD inhibitor.

The treatment of the cells with at least Wnt inhibitor can improve mDA neuron derivation. In certain embodiments, the treatment of the cells with at least Wnt inhibitor increases expression of at least one of A9 subtype mDA neuron markers, A10 subtype mDA neuron markers, and mDA neuron maturity markers. In certain embodiments, the treatment of the cells with at least Wnt inhibitor increases expression of ALDH1A1. In certain embodiments, the treatment of the cells with at least Wnt inhibitor increases expression CALB1. In certain embodiments, the treatment of the cells with at least Wnt inhibitor increases expression of DAT. In certain embodiments, the treatment of the cells with at least Wnt inhibitor increases expression of VMAT2. In certain embodiments, the treatment of the cells with at least Wnt inhibitor increases expressions of DAT and VMAT2.

In certain embodiments, at least about 50% (e.g., at least about 60%, at least about 70%, at least about 80%, at least about 85%, at least about 90%) of the differentiated cells express ALDH1A1 about 15 days from the initial contact of the stem cells with the at least one inhibitor of SMAD signaling. In certain embodiments, at least about 50% (e.g., at least about 60%, at least about 70%, at least about 80%, at least about 85%, at least about 90%) of the differentiated cells express ALDH1A1 16 days from the initial contact of the stem cells with the at least one inhibitor of SMAD signaling. In certain embodiments, at least about 50% (e.g., at least about 60%, at least about 70%, at least about 80%, at least about 85%, at least about 90%) of the differentiated cells express ALDH1A1 about 25 days from the initial contact of the stem cells with the at least one inhibitor of SMAD signaling. In certain embodiments, at least about 50% (e.g., at least about 60%, at least about 70%, at least about 80%, at least about 85%, at least about 90%) of the differentiated cells express ALDH1A1 about 30 days from the initial contact of the stem cells with the at least one inhibitor of SMAD signaling.

Furthermore, the mDA neurons or precursors thereof generated by the methods disclosed herein have improved fiber outgrowth, reduced remaining Ki67⁺ proliferating cells, and improved in vivo survival, which make these cells more suitable for therapeutic uses. In certain embodiments, the mDA neurons or precursors thereof generated by the methods disclosed herein have a detectable expression level of at least one mDA neuron marker at least about 2 weeks, at least about 3 weeks, at least about 1 month, at least about 2 months, at least about 3 months, at least about 4 months, at least about 5 months, at least about 6 months, at least about 1 year, at least about 2 years, at least about 3 years, at least about 4 years, or at least about 5 years post in vivo transplantation. In certain embodiments, the mDA neurons or precursors thereof generated by the methods disclosed herein have a detectable expression level of at least one mDA neuron marker at least about 2 weeks post in vivo transplantation. In certain embodiments, the mDA neurons or precursors thereof generated by the methods disclosed herein can have a detectable expression level of at least one mDA neuron marker up to about 1 month, up to about 2 months, up to about 3 months, up to about 4 months, up to about 5 months, up to about 6 months, up to about 1 year, up to about 2 years, up to about 3 years, up to about 4 years, or up to about 5 years post in vivo transplantation. In certain embodiments, the mDA neurons or precursors thereof generated by the methods disclosed herein have a detectable expression level of at least one mDA neuron marker about 1 month post in vivo transplantation. In certain embodiments, the mDA neurons or precursors thereof generated by the methods disclosed herein have a detectable expression level of at least one mDA neuron marker about 2 months post in vivo transplantation. In certain embodiments, the mDA neurons or precursors thereof generated by the methods disclosed herein have a detectable expression level of at least one marker selected from the group consisting of TH, EN1, NURR1, and ALDH1A1 at least about 1 month post in vivo transplantation. In certain embodiments, the mDA neurons or precursors thereof generated by the methods disclosed herein have a detectable expression level of at least one marker selected from the group consisting of TH, EN1, NURR1, and ALDH1A1 about 2 months post in vivo transplantation. In certain embodiments, the mDA neurons or precursors thereof generated by the methods disclosed herein have a detectable expression level of at least one marker selected from the group consisting of TH, EN1, NURR1, and ALDH1A1 at least about 2 months post in vivo transplantation.

In certain embodiments, the differentiated cells derived from the presently disclosed methods do not express or have a low expression of at least one marker selected from PAX6, EMX2, LHX2, SMA, SIX1, PITX2, SIM1, POU4F1, PHOX2A, BARHL1, BARHL2, GBX2, HOXA1, HOXA2, HOXB1, HOXB2, POU5F1, NANOG, and combinations thereof.

In certain embodiments, the cells are contacted with the activators and inhibitors described herein at a concentration and time effective to decrease expression of SMA, SIX1, PITX2, SIM1, POU4F1, and/or PHOX2A. In certain embodiments, the cells are contacted with the activators and inhibitors described herein at a concentration and time effective to decrease expression of PAX6, BARHL1, and/or BARHL2.

In certain embodiments, at least about 80% of the differentiated cells express FOXA2 and EN1 about 15 days from the initial contact of the stem cells with the at least one inhibitor of SMAD signaling. In certain embodiments, greater than about 80% (e.g., greater than about 85% or greater than about 90%) of the differentiated cells express FOXA2 and EN1 16 days from the initial contact of the stem cells with the at least one inhibitor of SMAD signaling.

### 5.2.10. Sorting Methods

In certain embodiments, the differentiation methods disclosed herein further comprise isolating mDA neurons and precursors thereof based on at least one or at least two surface markers. In certain embodiments, the surface marker is a negative surface marker, wherein the cells do not express a detectable level of the negative surface marker. In certain embodiments, the surface marker is a positive surface marker, wherein the cells express a detectable level of the positive surface marker.

In certain embodiments, the differentiation methods disclosed herein further comprise isolating cells that do not express a detectable level of at least one negative surface marker. In certain embodiments, the differentiation methods disclosed herein further comprise isolating cells that express a detectable level of at least one positive surface marker. In certain embodiments, the differentiation methods disclosed herein further comprise isolating cells that do not express a detectable level of at least one negative surface marker and express a detectable level of at least one positive surface marker.

In certain embodiments, the at least one negative surface marker is selected from the group consisting of CD49e, CD99, CD340, and combinations thereof. In certain embodiments, the at least one negative surface marker comprises CD49e. In certain embodiments, the at least one positive surface marker is selected from the group consisting of CD171, CD184, and combinations thereof. In certain embodiments, the at least one positive surface marker comprises CD184.

In certain embodiments, the differentiation methods disclosed herein further comprise isolating cells that do not express a detectable level of CD49e and express a detectable level of CD184.

Any surface-marker based cell isolation technology known in the art can be used in the presently disclosed methods. In certain embodiments, flow cytometry is used to the presently disclosed isolation methods.

### 5.2.11. Differentiation of mDA Precursors to mDA Neurons

In certain embodiments, the cells (e.g., mDA precursors) are further contacted with DA neuron lineage specific activators and inhibitors, for example, L-glutamine, brain-derived neurotrophic factor (BDNF), glial cell-derived neurotrophic factor (GDNF), Cyclic adenosine monophosphate (cAMP), Transforming growth factor beta (TGFβ, for example, TGFβ3), ascorbic acid (AA), and DAPT (which is also known as, N-[(3,5-Difluorophenyl)acetyl]-L-alanyl-2-phenyl]glycine-1,1-dimethylethyl ester; LY-374973, N—[N-(3,5-Difluorophenacetyl)-L-alanyl]-S-phenylglycine t-butyl ester; or N—[N-(3,5-difluorophenacetyl)-L-alanyl]-S-phenylglycine t-butyl ester). In certain embodiments, the cells are contacted with the foregoing DA neuron lineage specific activators and inhibitors for at least about 2, at least about 3, at least about 4, at least about 5, at least about 6, at least about 7, at least about 8, at least about 9, or at least about 10 or more days, for example, between about 2 days and about 20 days, between about 3 days and about 19 days, between about 4 days and about 18 days, between about 5 days and about 17 days, between about 6 days and about 16 days, between about 7 days and about 15 days, between about 8 days and about 15 days, between about 9 days and about 14 days, or between about 10 days and about 13 days. In certain embodiments, the cells are contacted with the foregoing DA neuron lineage specific activators and inhibitors for up to about 2, up to about 3, up to about 4, up to about 5, up to about 6, up to about 7, up to about 8, up to about 9, or up to about 10 days or more days. In certain embodiments, the cells are contacted with the foregoing DA neuron lineage specific activators and inhibitors for about 4 days, about 5 days, about 6 days, about 7 days, or about 8 days.

In certain embodiments, the cells are contacted with L-glutamine at a concentration of between about 0.5 mM and about 5 mM, or between about 1 mM and about 5 mM, or between about 1.5 mM and about 2.5 mM, or between about 1 mM and about 2 mM. In certain embodiments, the cells are contacted with L-glutamine at a concentration of about 2 mM.

In certain embodiments, the cells are contacted with BDNF at a concentration of between about 5 ng/ml and about 50 ng/mL, or between about 10 ng/ml and about 50 ng/mL, or between about 10 ng/ml and about 40 ng/mL, or between about 20 ng/ml and about 50 ng/mL, or between about 20 ng/ml and about 40 ng/mL, or between about 10 ng/ml and about 30 ng/mL, or between about 10 ng/ml and about 20 ng/mL, or between about 20 ng/ml and about 30 ng/mL. In certain embodiments, the cells are contacted with BDNF at a concentration of about 20 ng/mL.

In certain embodiments, the cells are contacted with ascorbic acid (AA) at a concentration of between about 50 nM and about 500 nM, or between about 100 nM and about 500 nM, or between about 100 nM and about 400 nM, or between about 200 nM and about 400 nM, or between about 200 nM and about 300 nM, or between about 100 nM and about 300 nM. In certain embodiments, the cells are contacted with AA at a concentration of about 200 nM.

In certain embodiments, the cells are contacted with GDNF at a concentration of between about 5 ng/ml and about 50 ng/mL, or between about 10 ng/ml and about 50 ng/mL, or between about 10 ng/ml and about 40 ng/mL, or between about 20 ng/ml and about 50 ng/mL, or between about 20 ng/ml and about 40 ng/mL, or between about 10 ng/ml and about 30 ng/mL, or between about 10 ng/ml and about 20 ng/mL, or between about 20 ng/ml and about 30 ng/mL. In certain embodiments, the cells are contacted with GDNF at a concentration of about 20 ng/mL.

In certain embodiments, the cells are contacted with cAMP at a concentration of between about 200 nM and about 800 nM, or between about 200 nM and about 700 nM, or between about 300 nM and about 700 nM, or between about 300 nM and about 600 nM, or between about 400 nM and about 600 nM, or between about 450 nM and about 550 nM. In certain embodiments, the cells are contacted with cAMP at a concentration of about 500 nM.

In certain embodiments, the cells are contacted with TGFβ3 at a concentration of between about 0.01 ng/ml and about 5 ng/mL, or between about 0.1 ng/ml and about 4 ng/mL, or between about 0.5 ng/ml and about 5 ng/mL, or between about 1 ng/ml and about 3 ng/mL, or between about 1 ng/ml and about 2 ng/mL. In certain embodiments, the cells are contacted with TGFβ3 at a concentration of about 1 ng/mL.

In certain embodiments, the cells are contacted with DAPT at a concentration of between about 1 nM and about 50 nM, or between about 5 nM and about 50 nM, or between about 1 nM and about 20 nM, or between about 5 nM and about 20 nM, or between about 1 nM and about 10 nM, or between about 5 nM and about 10 nM, or between about 5 nM and about 15 nM, or between about 10 nM and about 20 nM, or between about 10 nM and about 30 nM, or between about 30 nM and about 50 nM. In certain embodiments, the cells are contacted with DAPT at a concentration of about 10 nM.

In certain embodiments, the differentiated midbrain DA precursors are further cultured as described by U.S. Publication No. 2015/0010514, which is incorporated by reference in its entirety.

### 5.3. Cell Populations and Compositions

The presently disclosure provides a cell population of in vitro differentiated cells obtained by the methods disclosed herein, for example, in Section 5.2.

The presently disclosure provides a cell population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the cells express at least one marker indicating a mDA neuron or a precursor thereof. Non-limiting examples of markers indicating a mDA neuron or a precursor thereof include EN1, OTX2, TH, NURR1, FOXA2, LMX1A, PITX3, LMO3, SNCA, ADCAP1, CHRNA4, SOX6, ALDH1A1, WNT1, DAT, VMAT1, and GIRK2. The presently disclosure also provides compositions comprising such cell populations. In certain embodiments, the in vitro differentiated cells are obtained by the differentiation methods described herewith, for example, in Section 5.2.

In certain embodiments, less than about 50% (e.g., less than about 45%, less than about 40%, less than about 35%, less than about 30%, less than about 25%, less than about 20%, less than about 15%, less than about 10%, less than about 5%, less than about 4%, less than about 3%, less than about 2%, less than about 1%, less than about 0.5%, or less than about 0.1%) of the differentiated cells express at least one marker selected from PAX6, EMX2, LHX2, SMA, SIX1, PITX2, SIM1, POU4F1, PHOX2A, BARHL1, BARHL2, GBX2, HOXA1, HOXA2, HOXB1, HOXB2, POUSF1, NANOG, and combinations thereof.

In addition, the present disclosure provides compositions comprising any of the cell populations disclosed herein.

In certain embodiments, the cells are comprised in a composition that further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties). In certain embodiments, the composition further comprises growth factors for promoting maturation of the implanted/grafted cells into midbrain DA cells.

In certain embodiments, the composition comprises a cell population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the cells are administered to a subject. In certain embodiments, from about 1×10⁵ to about 1×10⁷ the cells thereof are administered to a subject.

In certain embodiments, said composition is frozen. In certain embodiments, said composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that comprises a pharmaceutically acceptable carrier. The compositions can be used for preventing and/or treating a neurodegenerative disorder include Parkinson's disease, Huntington's disease, Alzheimer's disease, and multiple sclerosis.

The presently disclosed subject matter also provides a device comprising the differentiated cells or the composition comprising thereof, as disclosed herein. Non-limiting examples of devices include syringes, fine glass tubes, stereotactic needles and cannulas.

### 5.4. Method of Preventing, Modeling, and/or Treating Neurological Disorders

The cell populations and compositions disclosed herein (e.g., those disclosed in Section 5.3) can be used for preventing, modeling, and/or treating at least a symptom in a subject having a neurological disorder. The presently disclosed subject matter provides for methods of preventing, modeling, and/or treating at least a symptom in a subject having a neurological disorder. In certain embodiments, the method comprises administering an effective amount of the presently disclosed stem-cell-derived mDA neurons or a composition comprising thereof into a subject suffering from a neurological disorder. In certain embodiments, the composition is a pharmaceutical composition further comprising a pharmaceutically acceptable carrier.

In certain embodiments, the neurological disorder is characterized by reduction of midbrain dopamine neuron function. The reduction of midbrain dopamine neuron function can be age related.

In certain embodiments, the symptom for a neurological disorder is selected from the group consisting of tremor, bradykinesia, flexed posture, postural instability, rigidity, dysphagia, and dementia.

Non-limiting examples of neurological disorders include Parkinsonism, Parkinson's disease, Huntington's disease, Alzheimer's disease, and multiple sclerosis. In certain embodiments, the neurological disorder is Parkinsonism or Parkinson's disease.

In certain embodiments, the neurological disorder is Parkinson's disease. Primary motor signs of Parkinson's disease include, for example, but not limited to, tremor of the hands, arms, legs, jaw and face, bradykinesia or slowness of movement, rigidity or stiffness of the limbs and trunk and postural instability or impaired balance and coordination.

In certain embodiments, the neurological disorder is a parkinsonism disease, which refers to diseases that are linked to an insufficiency of dopamine in the basal ganglia, which is a part of the brain that controls movement. Symptoms include tremor, bradykinesia (extreme slowness of movement), flexed posture, postural instability, and rigidity. Non-limiting examples of parkinsonism diseases include corticobasal degeneration, Lewy body dementia, multiple systematrophy, and progressive supranuclear palsy.

The cells or compositions can be administered or provided systemically or directly to a subject for preventing, modeling, and/or treating a neurological disorder. In certain embodiments, the cells or compositions are directly injected into an organ of interest (e.g., the central nervous system (CNS)). In certain embodiments, the cells or compositions are directly injected into the striatum.

The cells or compositions can be administered in any physiologically acceptable vehicle. The cells or compositions can be administered via localized injection, orthotopic (OT) injection, systemic injection, intravenous injection, or parenteral administration. In certain embodiments, the cells or compositions are administered to a subject suffering from a neurodegenerative disorder via orthotopic (OT) injection.

The cells or compositions can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived precursors. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the cells is the quantity of cells necessary to achieve an optimal effect. An optimal effect includes, but is not limited to, repopulation of CNS regions of a subject suffering from a neurodegenerative disorder, and/or improved function of the subject's CNS.

An “effective amount” (or “therapeutically effective amount”) is an amount sufficient to affect a beneficial or desired clinical result upon treatment. An effective amount can be administered to a subject in at least one doses. In terms of treatment, an effective amount is an amount that is sufficient to palliate, ameliorate, stabilize, reverse or slow the progression of the neurodegenerative disorder, or otherwise reduce the pathological consequences of the neurodegenerative disorder. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the cells is an amount that is sufficient to repopulate a CNS region of a subject suffering from a neurological disorder. In certain embodiments, an effective amount of the cells is an amount that is sufficient to improve the function of the CNS of a subject suffering from a neurodegenerative disorder, e.g., the improved function can be about 5%, about 10%, about 20%, about 30%, about 40%, about 50%, about 60%, about 70%, about 80%, about 90%, about 95%, about 98%, about 99% or about 100% of the function of a normal person's CNS.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the cells are administered to a subject. In certain embodiments, from about 1×10⁵ to about 1×10⁷ of the cells are administered to a subject suffering from a neurological disorder. In certain embodiments, from about 1×10⁶ to about 1×10⁷ of the cells are administered to a subject suffering from a neurological disorder. In certain embodiments, from about 1×10⁶ to about 4×10⁶ of the cells are administered to a subject suffering from a neurological disorder. The precise determination of what would be considered an effective dose may be based on factors individual to each subject, including their size, age, sex, weight, and condition of the particular subject. Dosages can be readily ascertained by those skilled in the art from this disclosure and the knowledge in the art.

### 5.5. Kits

The presently disclosed subject matter provides kits for inducing differentiation of stem cells to mDA neurons or precursors thereof. In certain embodiments, the kit comprises (a) at least one inhibitor of SMAD signaling, (b) at least one activator of Wnt signaling, (c) at least one activator of SHH signaling, (d) at least one activator of FGF signaling, and (e) at least one inhibitor of Wnt signaling. In certain embodiments, the kit further comprises (f) instructions for inducing differentiation of the stem cells into a population of differentiated cells that express at least one marker indicating a mDA neuron or a precursor thereof.

In certain embodiments, the instructions comprise contacting the stem cells with the inhibitor(s) and activator(s) in a specific sequence. The sequence of contacting the inhibitor(s) and activator(s) can be determined by the cell culture medium used for culturing the stem cells.

In certain embodiments, the instructions comprise contacting the stem cells with the inhibitor(s) and activator(s) as described by the methods of the present disclosure (see Section 5.2).

In certain embodiments, the present disclosure provides kits comprising an effective amount of a cell population or a composition disclosed herein in unit dosage form. In certain embodiments, the kit comprises a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit comprises instructions for administering the cell population or composition to a subject suffering from a neurological disorder. The instructions can comprise information about the use of the cells or composition for preventing, modeling, and/or treating at least a symptom in a subject having a neurological disorder. In certain embodiments, the instructions comprise at least one of the following: description of the therapeutic agent; dosage schedule and administration for preventing, modeling, and/or treating at least a symptom in a subject having a neurological disorder or symptoms thereof, precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

### 6. EXAMPLES

The presently disclosed subject matter will be better understood by reference to the following Example, which is provided as exemplary of the presently disclosed subject matter, and not by way of limitation.

### Example 1: Exemplary Midbrain DA Neuron Differentiation Protocol

The following is an exemplary protocol of the presently disclosed method in accordance with certain embodiments.

Day 0: Cells were fed with Accutase from hPSC/hiPSC at single cells and plate at a density of 400,000 cells/cm² on Geltrex-coated plated in Medium 1 with Y-drug.

Day 1-Day 2: cells should have reached 100% confluence. Cells were double fed with Medium 1.

Day 3: Cells were fed with Medium 1.

Day 4: Cells were fed with Medium 2. For CHIR-Boost protocol, CHIR concentration was changed from 1 μM to 6 μM for WA-09 hESC line-mediated differentiation (this can be slightly vary depending on hPSC/hiPSC lines).

Day 5-Day 6: Cells were double fed with Medium 2.

Day 7: Cells were fed with Medium 3.

Day 8-Day 9: Cells were fed daily with Medium 3.

Day 10: Cells were fed with Medium 4.

Day 11: Cells were incubated with Accutase for 30 minutes at 37 C; plate cells at a density of 800,000 cells/cm² in Medium 4.

Day 12: Cells should have reached 100% confluence. Cells were fed with Medium 5.

Day 12-16: Cells were fed daily with Medium 5; at day 16, more than 90% of cells were FOXA2⁺/EN⁺, as measured by FACS analysis.

Day 16-Day 100: Cells were fed daily with Medium 6.

Medium 1 composition: neurobasal medium, N2 supplement, B27 supplement, Pen/Strep, L-Glutamine, 10 μM SB, 250 nM LDN, 500 ng/ml SHH C5II, and 1 μM CHIR.

Medium 2 composition: neurobasal medium, N2 supplement, B27 supplement, Pen/Strep, L-Glutamine, 10 mM SB, 250 nM LDN, 500 ng/ml SHH C5II, and 6 μM CHIR.

Medium 3 composition: neurobasal medium, N2 supplement, B27 supplement, Pen/Strep, L-Glutamine, and 6 μM CHIR.

Medium 4 composition: neurobasal medium, B27 supplement, Pen/Strep, L-Glutamine, 3 μM CHIR, 20 ng/ml BDNF, 0.2 μM ascorbic acid (AA), 20 ng/ml GSNF, 0.5 mM dcAMP, and 1 ng/ml TGF-β3.

Medium 5 composition: neurobasal medium, B27 supplement, Pen/Strep, L-Glutamine, 3 μM CHIR, 20 ng/ml BDNF, 0.2 μM AA, 20 ng/ml GDNF, 0.5 mM dcAMP, 1 ng/ml TGF-β3, 1 μM IWP2, and 100 ng/ml FGF18.

Medium 6 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, and 10 nM DAPT.

The protocol described in this Example is referred to as “Wnt-boost+FGF18 (day 12-day 16)+IWP2 (day 12-day 16)” in Example 3.

### Example 2: Exemplary Midbrain DA Neuron Differentiation Protocol

The following is an exemplary protocol of the presently disclosed method in accordance with certain embodiments.

Day 0: Cells were fed with Accutase from hPSC/hiPSC at single cells and plate at a density of 400,000 cells/cm² on Geltrex-coated plated in Medium 1 with Y-drug.

Day 1-Day 2: cells should have reached 100% confluence. Cells were double fed with Medium 1.

Day 3: Cells were fed with Medium 1.

Day 4: Cells were fed with Medium 2. For CHIR-Boost protocol, CHIR concentration was changed from 1 μM to 6 μM for WA-09 hESC line-mediated differentiation (this can be slightly vary depending on hPSC/hiPSC lines).

Day 5-Day 6: Cells were double fed with Medium 2.

Day 7: Cells were fed with Medium 3.

Day 8-Day 9: Cells were fed daily with Medium 3.

Day 10: Cells were fed with Medium 4.

Day 11: Cells were incubated with Accutase for 30 minutes at 37 C; plate cells at a density of 800,000 cells/cm² in Medium 4.

Day 12: Cells should have reached 100% confluence. Cells were fed with Medium 5.

Day 12-16: Cells were fed daily with Medium 5; at day 16, more than 90% of cells were FOXA2⁺/EN⁺, as measured by FACS analysis.

Day 16-Day 100: Cells were fed daily with Medium 6.

Medium 1 composition: neurobasal medium, N2 supplement, B27 supplement, Pen/Strep, L-Glutamine, 10 μM SB, 250 nM LDN, 500 ng/ml SHH C25II, and 1 μM CHIR.

Medium 2 composition: neurobasal medium, N2 supplement, B27 supplement, Pen/Strep, L-Glutamine, 10 mM SB, 250 nM LDN, 500 ng/ml SHH C25II, and 6 μM CHIR.

Medium 3 composition: neurobasal medium, N2 supplement, B27 supplement, Pen/Strep, L-Glutamine, and 6 μM CHIR.

Medium 4 composition: neurobasal medium, B27 supplement, Pen/Strep, L-Glutamine, 1 μM IWP2, 20 ng/ml BDNF, 0.2 μM ascorbic acid (AA), 20 ng/ml GDNF, 0.5 mM dcAMP, and 1 ng/ml TGF-β3.

Medium 5 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, 1 μM IWP2, and 100 ng/ml FGF18.

Medium 6 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, and DAPT (10 nM).

The protocol described in this Example is referred to as “Wnt-boost+FGF18 (day 12-day 16)+IWP2 (day 10-day 16)” in Example 3.

### Example 3: Wnt Inhibitor Treatment During mDA Neuron Differentiation

Different WNT signaling genes are linked to dopamine neuron subtypes. Aldehyde Dehydrogenase 1 Family Member A1 (ALDH1A1) is a marker for hDA2 subtype (A9 type) during mouse and human mDA neuron development ((La Manno, et al. Cell 167, 566-580 e519 (2016); Toledo, et al. Br J Pharmacol 174(24), 4716-4724 (2017)). ALDH1A1 belongs to the aldehyde dehydrogenases family of proteins and is the second enzyme of the major oxidative pathway of alcohol metabolism.

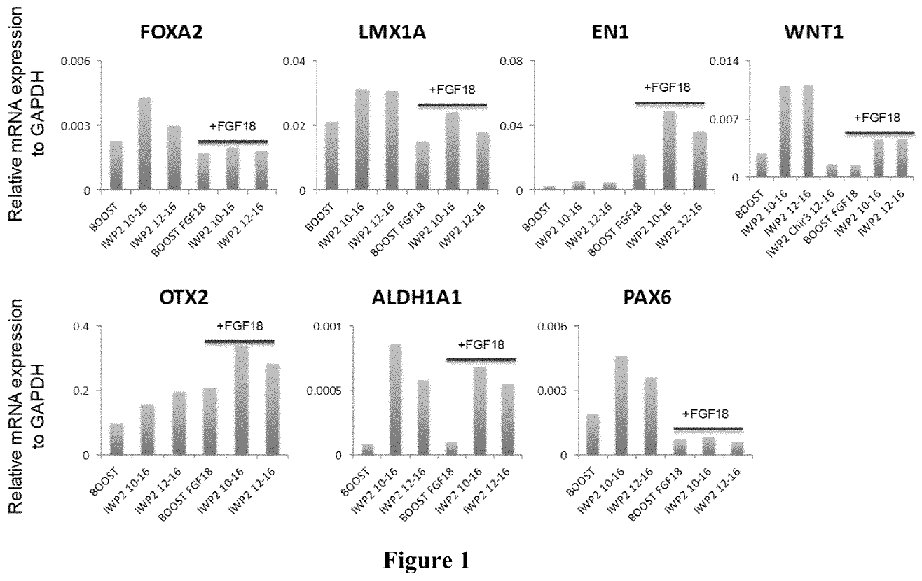

hPSC and hiPSC were used for the differentiation methods. First, the effect of Wnt signaling on ALDH1A1 induction in mDA cells differentiated with different protocols was evaluated. The mRNA expression levels of FOXA2, LMX1A, EN1, WNT1, OTX2, ALDH1A1 and PAX6 were evaluated in day 16-differentiated mDA cells produced using Wnt-boost, Wnt-boost+IWP2 (day 10-day 16), and Wnt-boost+IWP2 (day 12-day 16) protocols, with or without FGF18 (day 12-16) (FIG. 1).

The “Wnt-boost+FGF18 (day 12-16)+IWP2 (day 12-day 16)” protocol is described in Example 1.

The “Wnt-boost+FGF18 (day 12-16)+IWP2 (day 10-day 16)” protocol is described in Example 2.

The “Wnt-boost” protocol referred to in this Example is provided below.

Day 0: Cells were fed with Accutase from hPSC/hiPSC at single cells and plate at a density of 400,000 cells/cm² on Geltrex-coated plated in Medium 1 with Y-drug.

Day 1-Day 2: cells should have reached 100% confluence. Cells were double fed with Medium 1.

Day 3: Cells were fed with Medium 1.

Day 4: Cells were fed with Medium 2. For CHIR-Boost protocol, CHIR concentration was changed from 1 μM to 6 μM for WA-09 hESC line-mediated differentiation (this can be slightly vary depending on hPSC/hiPSC lines).

Day 5-Day 6: Cells were double fed with Medium 2.

Day 7: Cells were fed with Medium 3.

Day 8-Day 9: Cells were fed daily with Medium 3.

Day 10: Cells were fed with Medium 4.

Day 11: Cells were incubated with Accutase for 30 minutes at 37 C; plate cells at a density of 800,000 cells/cm² in Medium 4.

Day 12: Cells should have reached 100% confluence. Cells were fed with Medium 5.

Day 12-16: Cells were fed daily with Medium 5; at day 16, more than 90% of cells were FOXA2⁺, as measured by FACS analysis.

Day 16-Day 100: Cells were fed daily with Medium 6.

Medium 1 composition: neurobasal medium, N2 supplement, B27 supplement, Pen/Strep, L-Glutamine, 10 μM SB, 250 nM LDN, 500 ng/ml SHH C25II, and 1 μM CHIR.

Medium 2 composition: neurobasal medium, N2 supplement, B27 supplement, Pen/Strep, L-Glutamine, 10 mM SB, 250 nM LDN, 500 ng/ml SHH C25II, and 6 μM CHIR.

Medium 3 composition: neurobasal medium, N2 supplement, B27 supplement, Pen/Strep, L-Glutamine, and 6 μM CHIR.

Medium 4 composition: neurobasal medium, B27 supplement, Pen/Strep, L-Glutamine, 3 μM CHIR, 20 ng/ml BDNF, 0.2 μM ascorbic acid (AA), 20 ng/ml GDNF, 0.5 mM dcAMP, and 1 ng/ml TGF-β3.

Medium 5 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3.

Medium 6 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, and 10 nM DAPT.

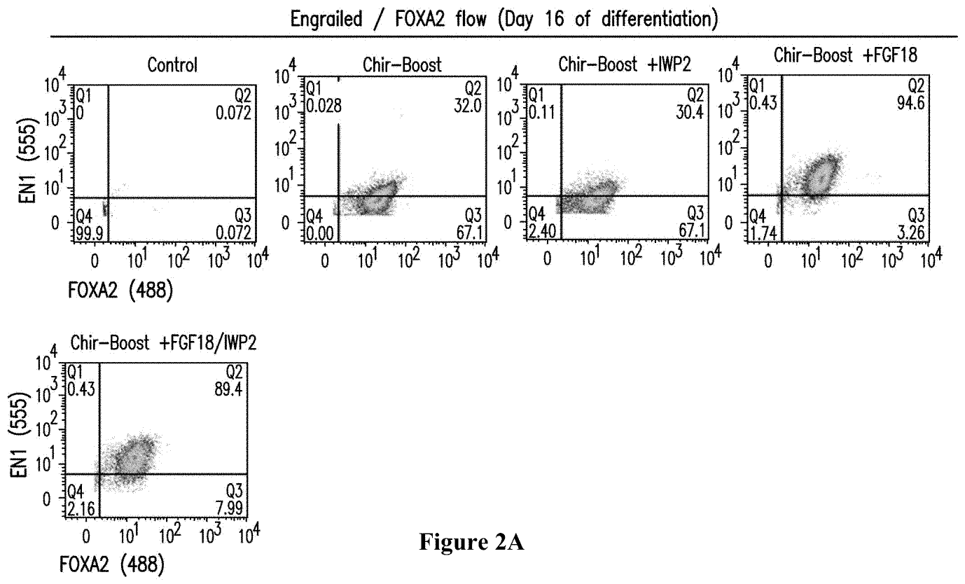

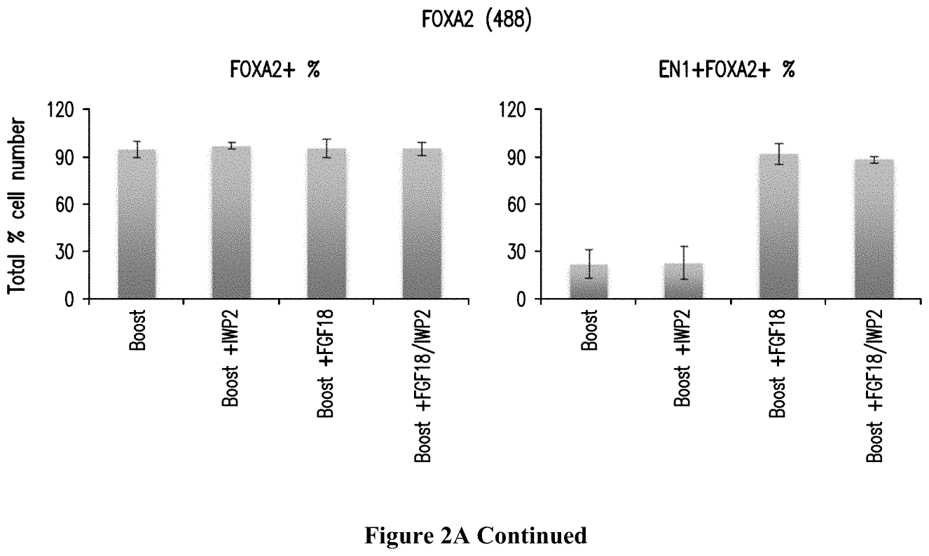

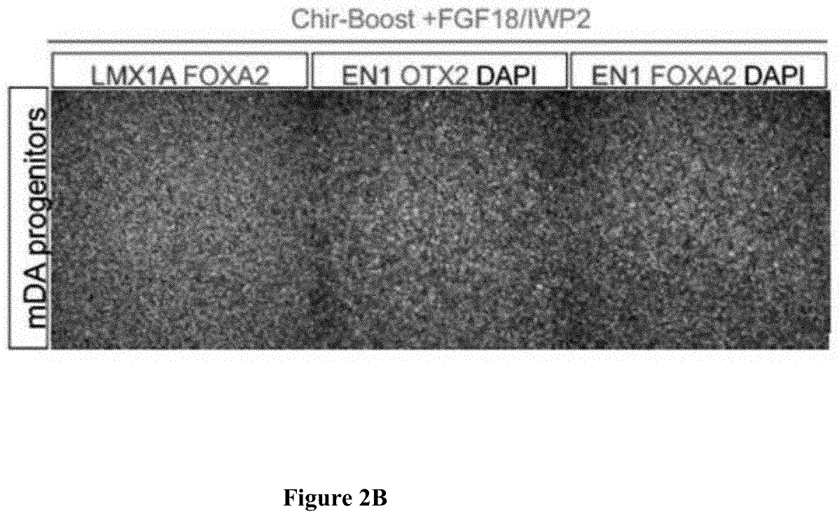

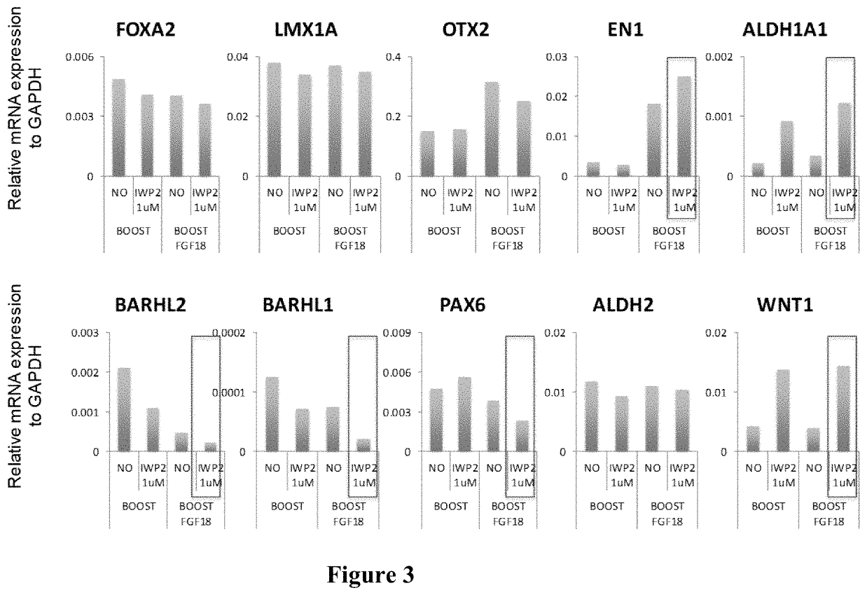

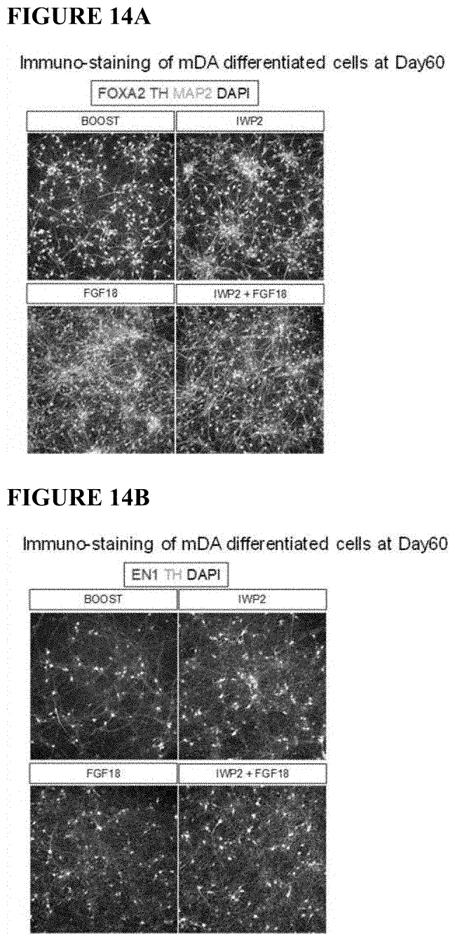

SMA mRNA expression level was not detectable. The Wnt-boost protocol in combination with FGF18 and IWP2 generated optimal A/P and DN patterned precursors, where more than 90% cells are FOXA2/EN1 double positive (FIG. 2A). FIG. 2A shows FACS analysis of day 16-differentiated mDA precursors using different protocols. Immune-staining images of day 16-differentied mDA using different protocols are showed in FIG. 2B. Furthermore, The mRNA expression levels of FOXA2, LMX1A, OTX2, EN1, ALDH1A1, BARHL2, BARHL1, PAX6, ALDH2, and WNT1 were evaluated at day 16 in differentiated mDA cells produced using Wnt-boost, Wnt-boost+IWP2 (day 12-day 16) protocols, with or without FGF18 (FIG. 3). The effect of IWP2 on the expression of marker genes in the differentiated cells was determined. As shown in FIG. 4, the mRNA expression levels of FOXA2, LMX1A, OTX2, EN1, ALDH1A1, PAX6 and PITX3 were evaluated in day-40 differentiated cells using Wnt-boost protocol, with or without the addition of FGF18 and/or IWP2 from day 12 to day 16. The present disclosure observed the presence of very high quadrupole positive cells at day 16 (FOXA2/LMX1A, OTX2/EN1). Furthermore, the high expression of EN1 was driven by FGF18. Additionally, the expression of ALDH1A1, WNT1, PITX3, DAT, DDC, VMTA2 were increased by the addition of IWP2 and FGF18, whereas IWP2 lowered the expression of Ki67, SMA and SIX1. Immuno-staining images of day 60-differentiated cells were collected showing the expression of FOXA2, TH, and MAP2 (FIG. 14A), and EN1 and TH (FIG. 14B).

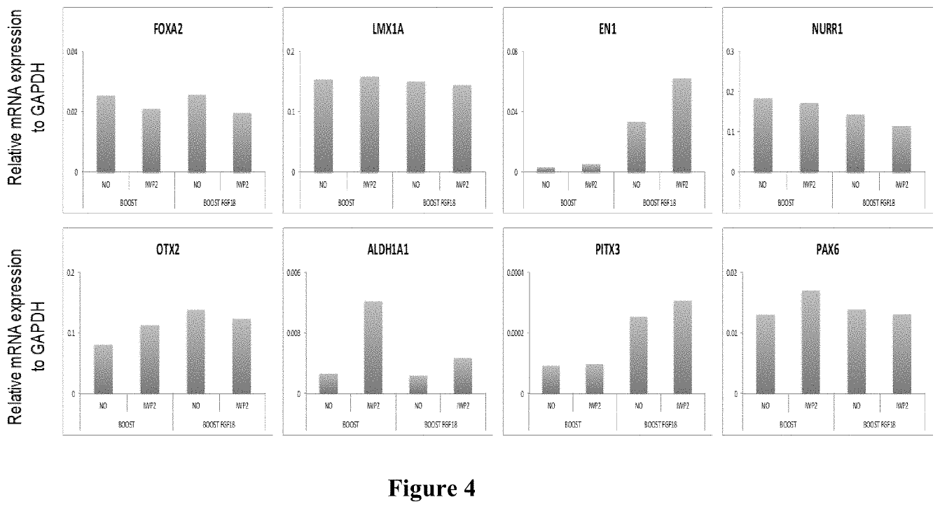

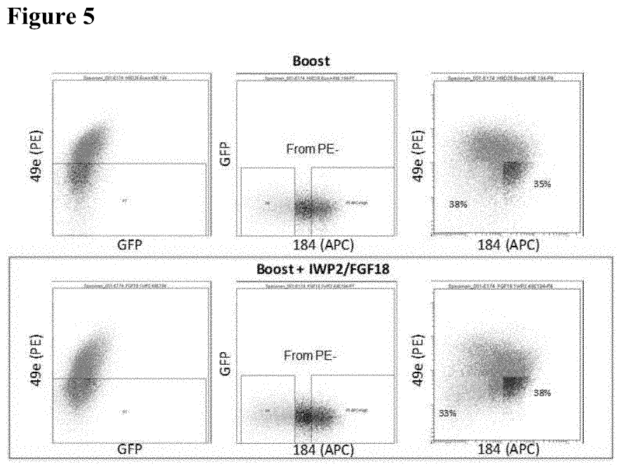

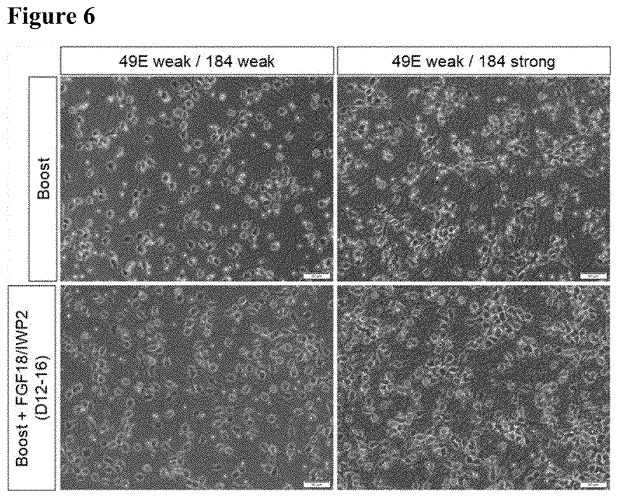

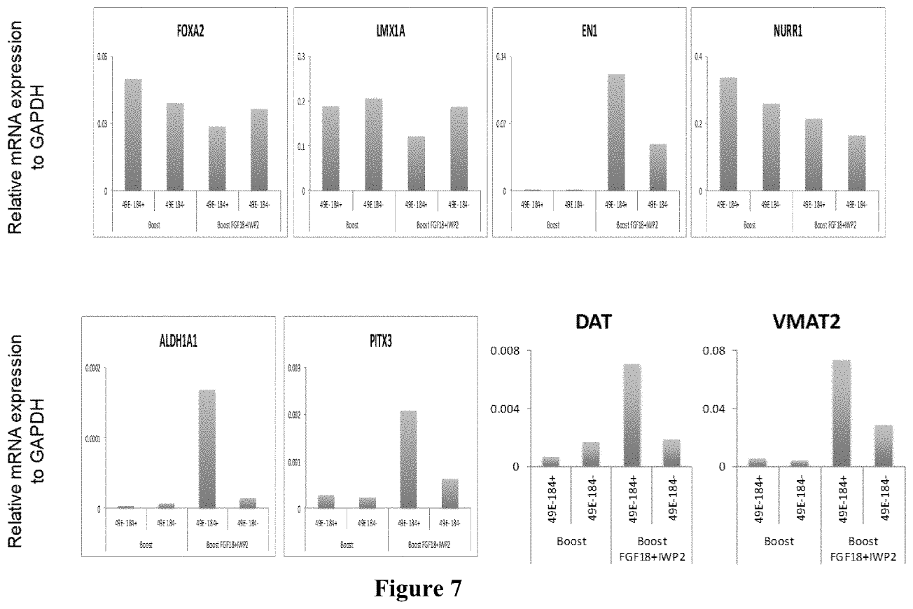

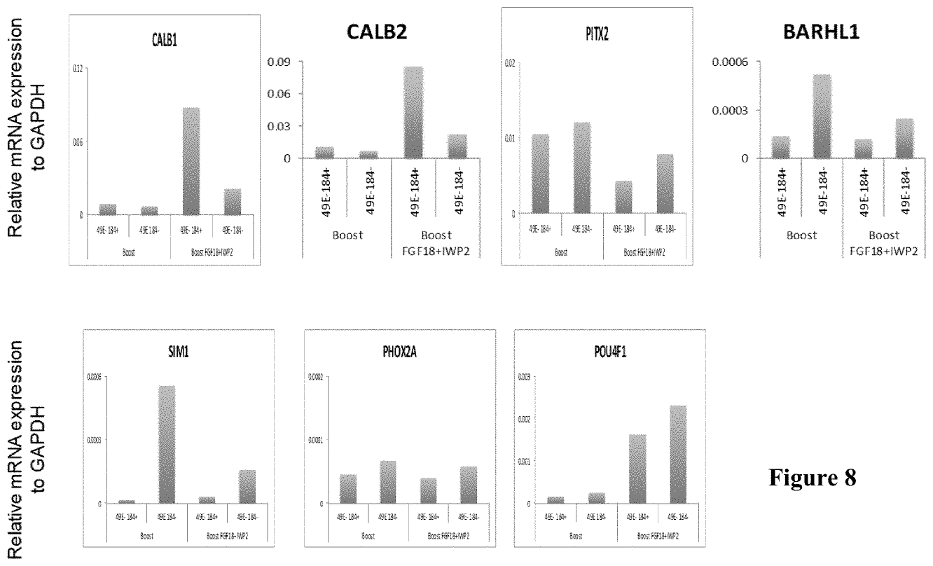

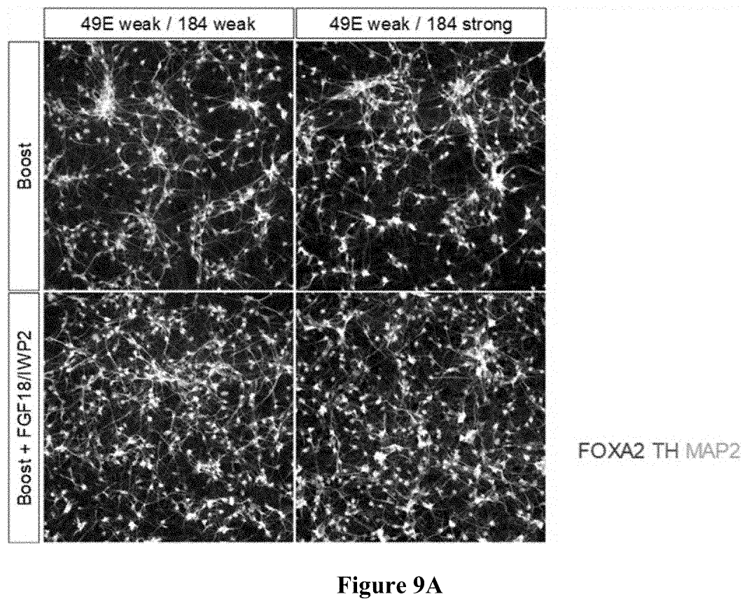

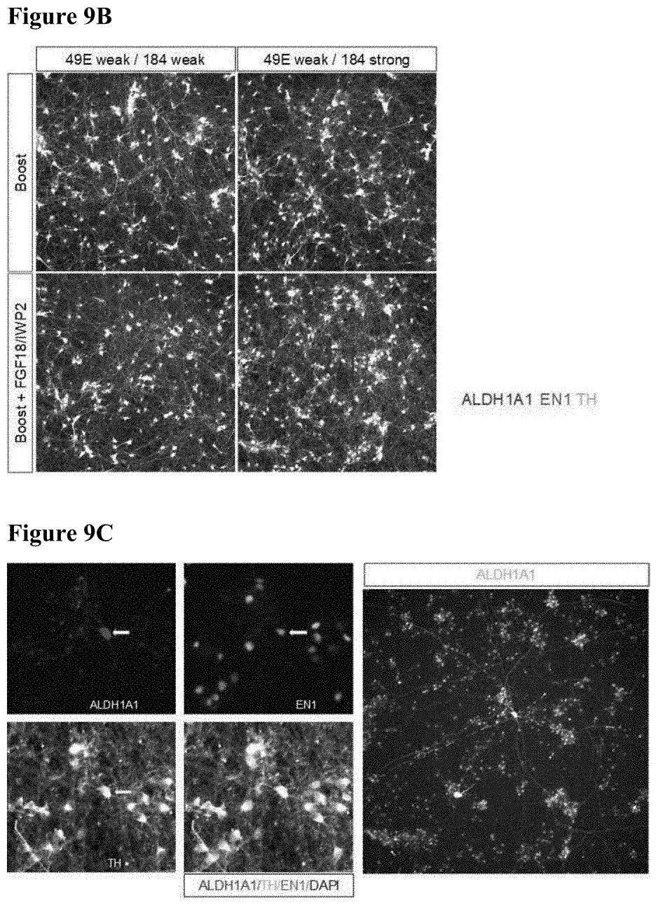

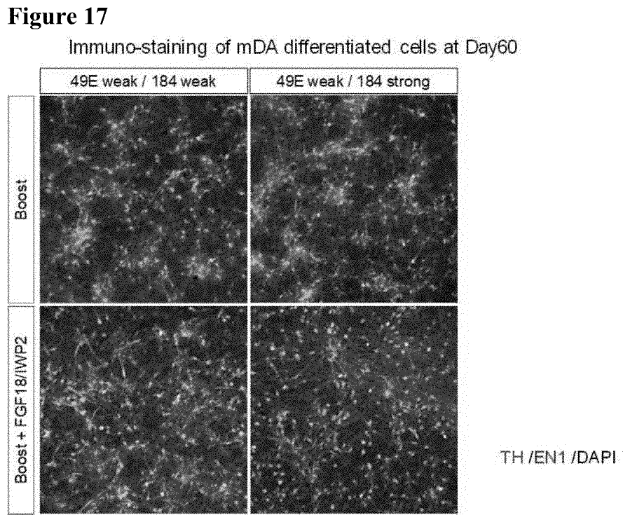

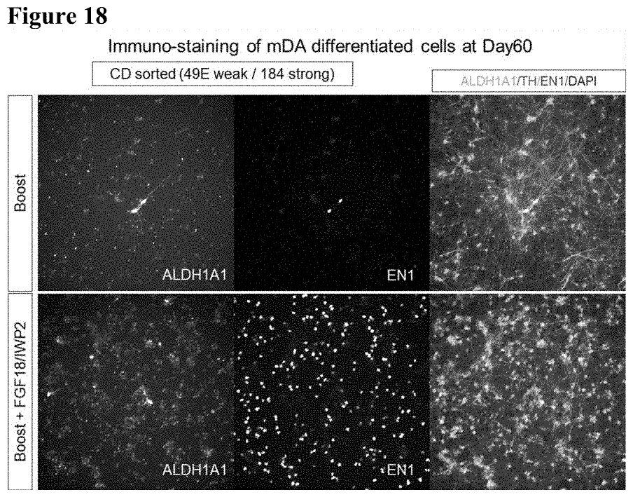

FACS-mediated sorting of day 25-differentiated cells using Wnt-boost protocol, with or without the addition of FGF18 and IWP2 was performed (FIG. 5). The differentiated mDA cells were sorted based on the expression of CD49e and CD184 protein markers. The morphology of the sorted day 40-differentiated CD49^{weak}/CD184^{weak} cells and CD49^{weak}/CD184^{strong} cells is shown in FIG. 6. The mRNA expression levels of FOXA2, LMX1A, EN1, NURR1, ALDH1A1, PITX3, DAT, VMAT2, CALB1, CALB2, PITX2, BARHL1, SIM1, PHOX2A, POU4F1 were evaluated in sorted day 40-differentiated CD49^{weak}/CD184^{weak} cells and CD49^{weak}/CD184^{strong} cells was analyzed (FIGS. 7 and 8). Immuno-staining images of sorted day 40-differentiated CD49^{weak}/CD184^{weak} cells and CD49^{weak}/CD184^{strong} cells were collected showing the expression of FOXA2, TH, and MAP2 (FIG. 9A) and ALDHA1A1, EN1, and TH (FIGS. 9B-9C). Furthermore, immuno-staining images of sorted day 60-differentiated CD49^{weak}/CD184^{weak} cells and CD49^{weak}/CD184^{strong} cells were collected showing the expression of TH and EN1 (FIG. 17) and ALDHA1A1, EN1, and TH (FIG. 18).

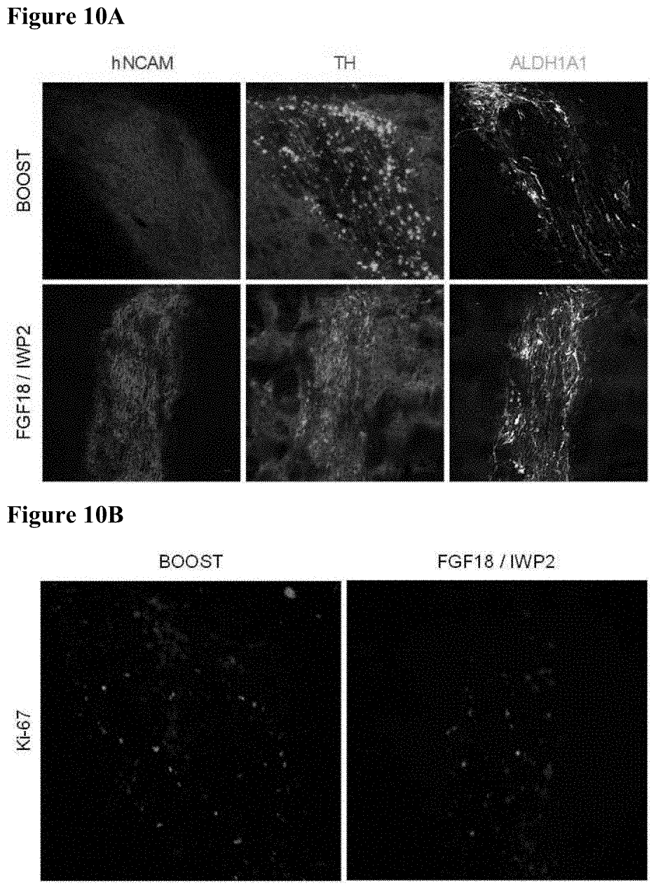

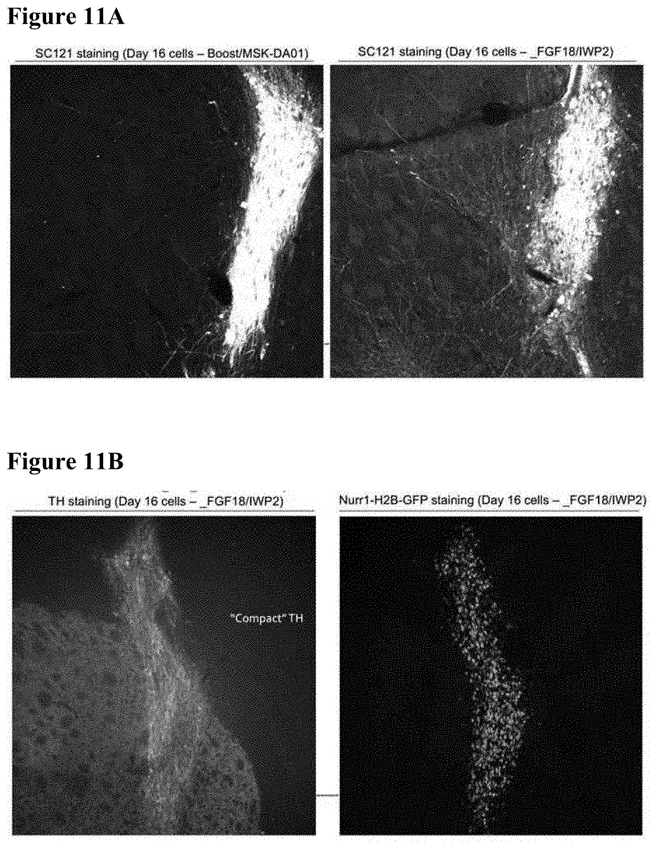

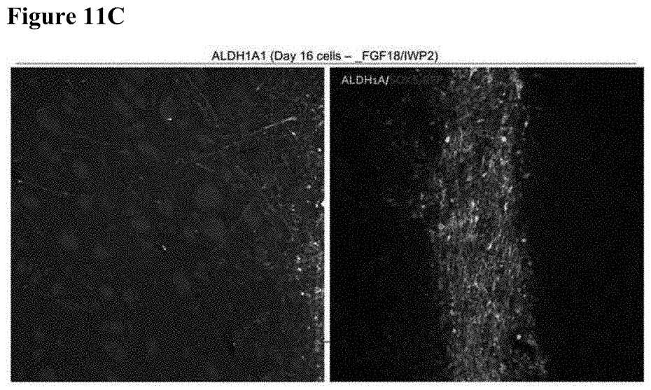

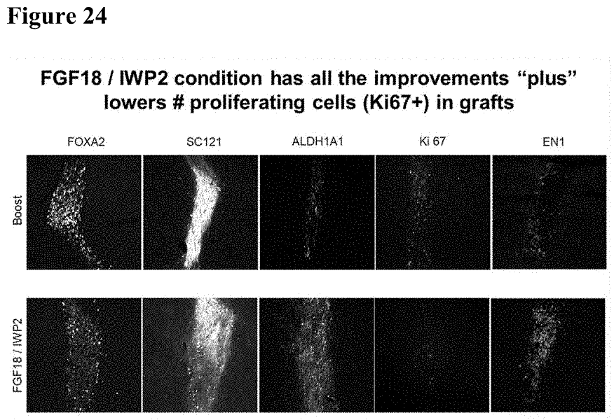

Next, cells were differentiated using “Wnt-boost” protocol or “Wnt-boost+FGF18 (day 12-16)+IWP2 (day 12-day 16)” protocol, and were transplanted in mice. Grafted cells were immune-stained one month after transplantation. The expression of hNCAM, TH, ALDH1A1, FOXA2, SC121, EN1, Ki67 were evaluated (FIGS. 10A, 10B, and 24). Additionally, grafted cells were immune-stained two months after transplantation. The expression levels of SC121, TH, Nurr1, ALDH1A1, and SOX6 were determined (FIGS. 11A-11C).

### Example 4: Optimizing WNT Inhibition

This example is designed to optimize the temporal window and concentration of the WNT treatment, and to test whether reactivation of non-canonical signaling is required for optimal levels of mDA neuron differentiation or maturation. Preliminary data (not shown) suggest that inhibition of canonical signaling alone (e.g., by using a selective inhibitor of canonical signaling (e.g., XAV939; tankyrase inhibitor stabilizing AXIN)) might not be sufficient to obtain comparable results to the IWP2 treatment. IWP2 inhibits both non-canonical signaling and canonical signaling. The expressions of PITX3, DAT, and VMAT2 are quantified by qRT-PCR and immunocytochemistry (ICC). It is confirmed that IWP2 (or other candidate WNT inhibitors) does not negatively affect the expression of EN1 or the emergence of contaminating markers (SIX1 and SMA). Optimized conditions are validated across hPSC lines (3 hESC and 3 iPSC lines; >3 independent differentiations each) (Zimmer, et al. Proceedings of the National Academy of Sciences of the United States of America 115, E8775-E8782 (2018)) including male and female lines.

### Example 5: In Vitro Detailed Molecular and Functional Assessment of Generated mDA Neurons

mDA neuron identity generated by the presently disclosed differentiation method is validated. This validation includes i) in-depth temporal characterization of marker expression (including ALDH1A1 and PITX3) by ICC and by in situ expression; ii) analysis of time course bulk RNAseq (day 0, day 11, day 16, day 25, day 40, day 60); iii) use of a set of 42 arrayed qRT-PCR markers developed for optimizing clinical-grade mDA neuron differentiation to assess whether the presently disclosed differentiation method matches or exceeds previously established QC (“release criteria”) for clinical-grade mDA neurons; iv) assessment of the biochemical maturity of mDA neurons by measuring DA release by HPLC (electrochemical detection) as described previously (Kriks, et al. Nature 480, 547-551 (2011)) at day 30, 50, and 70 of differentiation; and v) determination of differences in levels of maturity (e.g. resting membrane potential, input resistance) by in vitro electrophysiologic studies, and in mDA neuron specific parameters including autonomous pace-making or the presence of Sag current. It is expected that KCL-evoked DA released in mDA neurons developed by the presently disclosed differentiation method occurs earlier than prior methods and at higher levels on a per cell basis. The emergence of spontaneous in vitro network activity is validated using a high-density micro-electrode array system (MEA).

### Example 6: In Vivo Functional Evaluation of the Generated mDA Neurons

To assess in vivo survival and function, cells are transplanted on day 16. Short-term transplantations (1 month) are performed into the striatum of un-lesioned NSG mice to confirm robust short-term survival for each of the treatment groups prior to initiating functional studies (n=5/group). For functional studies, 6 months transplantation studies are run in 6OHDA lesioned rat hosts (nu/nu rat). The groups are i) saline control, ii) Wnt-Boost, iii) Wnt-Boost+FGF18, iv) Wnt-Boost+FGF18/IWP2 (n=10/group). Rats are subjected to unilateral 6OHDA lesioning targeting the medial forebrain bundle (MFB) prior to transplantation, as described previously (Kriks, et al. Nature 480, 547-551 (2011)). Only animals with stable rotation behavior (>6 rotations/min; 2 sequential tests at weekly intervals) are included. In addition to amphetamine-induced rotations (at monthly intervals), several non-drug induced assays including stepping and cylinder test (Kriks, et al. Nature 480, 547-551 (2011)) (prior to grafting and at 3 and 6 months post grafting) are monitored. Transplantation is performed via stereotactic surgery and injection of 200×10³ cells (2 μl volume) into the host striatum as described previously (Kriks, et al. Nature 480, 547-551 (2011)). It is expected that all conditions trigger significant recovery (compared to saline control) in amphetamine-induced behavior, while FGF18 and FGF18/IWP2 protocols trigger a more rapid recovery and possibly show overcompensation in the rotation assay (negative scores) at late time points. Furthermore, grafts of mDA neurons generated by the presently disclosed differentiation methods show enhanced recovery in stepping and cylinder tests, which are typically more challenging to restore than amphetamine-rotations.

### Example 7: Histological Analysis

Histological analysis is performed to address whether there are differences in i) the total number of surviving mDA neurons (stereological counts of TH+ cells in graft); ii) the human identity of TH+ cells confirmed by co-expression with human nuclear antigen (hNA); iii) markers of mDA neuron identity, subtype and biochemical maturation (TH/EN1/FOXA2, TH/DAT/VMAT2, TH/GIRK2/CALB); iv) extent of fiber outgrowth (% of striatal reinnervation by TH/hNCAM and or TH/SC121); v) percentage of non-dopamine neurons (GABA, Serotonin, Glutamate)11 and vi) the percentage of glial cells (GFAP, Olig2) and other proliferating (Ki67) cells.

### Example 8: Wnt Inhibitor Treatment During mDA Neuron Differentiation

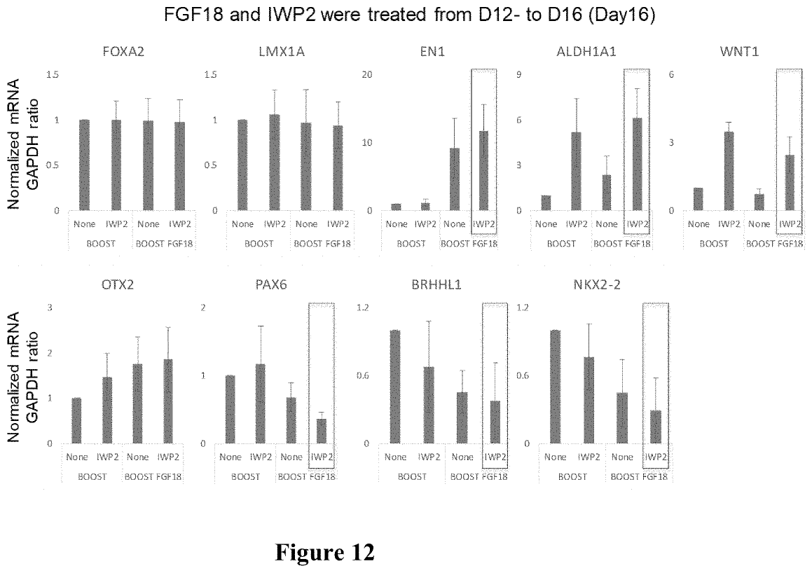

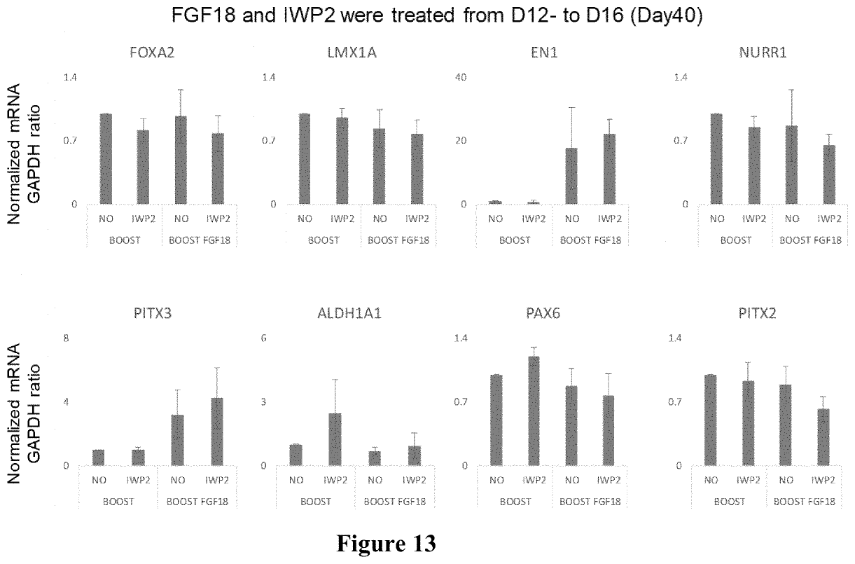

This Example shows updated experiments of Example 3. The mRNA expression levels of FOXA2, LMX1A, OTX2, EN1, ALDH1A1, WNT1, BARHL1, PAX6, OTX2, and NKX2-2 were evaluated at day 16 in differentiated mDA cells produced using Wnt-boost, Wnt-boost+IWP2 (day 12-day 16) protocols, with or without FGF18. The present disclosure discovered that IWP2 exposure led to increased ALDH1A1 as well as high endogenous WNT1 expression at day 16 both in Wnt Boost and Wnt Boost+FGF18 conditions. Moreover, IWP2 exposure lowered PAX6 and NKX2-2 expression (FIG. 12). Similar changes were observed in day 40-differentiated cells (FIG. 13). Immuno-staining of day 60-differentiated cells, which were produced using Wnt-boost protocol, with or without the addition of FGF18 and/or IWP2 from day 12 to day 16, confirmed that FGF18 and IWP2 exposure maintained the high proportion of FOXA2 and TH expression (FIG. 14A) and increased the expression of EN1 and TH in cells (FIG. 14B).

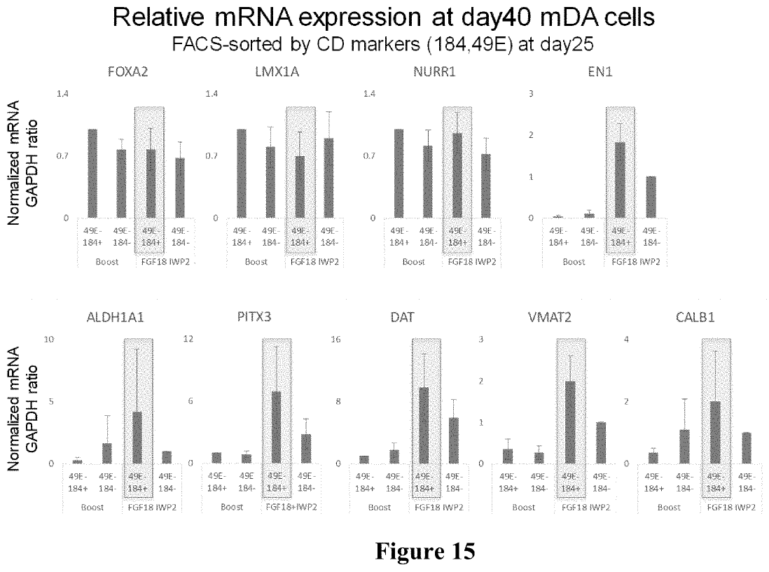

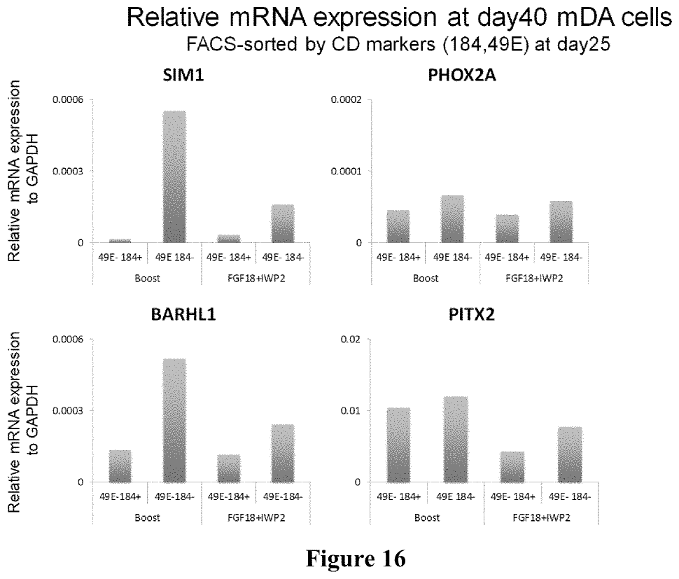

FACS-mediated sorting of day 25-differentiated cells using Wnt-boost protocol, with or without the addition of FGF18 and IWP2 was performed. The differentiated mDA cells were sorted based on the expression of CD49e and CD184 protein markers. The mRNA expression levels of FOXA2, LMX1A, EN1, NURR1, ALDH1A1, PITX3, DAT, VMAT2, CALB1, PITX2, BARHL1, SIM1, and PHOX2A were evaluated in sorted day 40-differentiated CD49^{weak}/CD184^{weak} cells and CD49^{weak}/CD184^{strong} cells (FIGS. 15 and 16). Immuno-staining images of sorted day 60-differentiated CD49^{weak}/CD184^{weak} cells and CD49^{weak}/CD184^{strong} cells showed the expression of ALDH1A1, EN1, and TH (FIGS. 17 and 18).

### Example 9: Increased Exposure to Wnt Inhibitors

Increased exposure to Wnt inhibitors was tested. An exemplary midbrain DA neuron differentiation protocol with Wnt inhibitor exposure from day 12 to day 25 is as the following:

Day 0: Cells were fed with Accutase from hPSC/hiPSC at single cells and plate at a density of 400,000 cells/cm² on Geltrex-coated plated in Medium 1 with Y-drug.

Day 1-Day 2: cells should have reached 100% confluence. Cells were double fed with Medium 1.

Day 3: Cells were fed with Medium 1.

Day 4: Cells were fed with Medium 2. For CHIR-Boost protocol, CHIR concentration was changed from 1 μM to 6 μM for WA-09 hESC line-mediated differentiation (this can be slightly vary depending on hPSC/hiPSC lines).

Day 5-Day 6: Cells were double fed with Medium 2.

Day 7: Cells were fed with Medium 3.

Day 8-Day 9: Cells were fed daily with Medium 3.

Day 10: Cells were fed with Medium 4.

Day 11: Cells were incubated with Accutase for 30 minutes at 37 C; plate cells at a density of 800,000 cells/cm² in Medium 4.

Day 12: Cells should have reached 100% confluence. Cells were fed with Medium 5.

Day 12-16: Cells were fed daily with Medium 5; at day 16, more than 90% of cells were FOXA2⁺/EN⁺, as measured by FACS analysis.

Day 16-Day 25: Cells were fed daily with Medium 6.

Day 25-Day 100: Cells were fed daily with Medium 7.

Medium 1 composition: neurobasal medium, N2 supplement, B27 supplement, Pen/Strep, L-Glutamine, 10 μM SB, 250 nM LDN, 500 ng/ml SHH C25II, and 1 μM CHIR.

Medium 2 composition: neurobasal medium, N2 supplement, B27 supplement, Pen/Strep, L-Glutamine, 10 mM SB, 250 nM LDN, 500 ng/ml SHH C25II, and 6 μM CHIR.

Medium 3 composition: neurobasal medium, N2 supplement, B27 supplement, Pen/Strep, L-Glutamine, and 6 μM CHIR.

Medium 4 composition: neurobasal medium, B27 supplement, Pen/Strep, L-Glutamine, 1 μM IWP2, 20 ng/ml BDNF, 0.2 μM ascorbic acid (AA), 20 ng/ml GDNF, 0.5 mM dcAMP, and 1 ng/ml TGF-β3.

Medium 5 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, 1 μM IWP2, and 100 ng/ml FGF18.

Medium 6 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, 1 μM IWP2, and DAPT (10 nM).

Medium 7 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, and DAPT (10 nM).

An exemplary midbrain DA neuron differentiation protocol with Wnt inhibitor exposure from day 12 to day 30 is as the following:

Day 0: Cells were fed with Accutase from hPSC/hiPSC at single cells and plate at a density of 400,000 cells/cm² on Geltrex-coated plated in Medium 1 with Y-drug.

Day 1-Day 2: cells should have reached 100% confluence. Cells were double fed with Medium 1.

Day 3: Cells were fed with Medium 1.

Day 4: Cells were fed with Medium 2. For CHIR-Boost protocol, CHIR concentration was changed from 1 μM to 6 μM for WA-09 hESC line-mediated differentiation (this can be slightly vary depending on hPSC/hiPSC lines).

Day 5-Day 6: Cells were double fed with Medium 2.

Day 7: Cells were fed with Medium 3.

Day 8-Day 9: Cells were fed daily with Medium 3.

Day 10: Cells were fed with Medium 4.

Day 11: Cells were incubated with Accutase for 30 minutes at 37 C; plate cells at a density of 800,000 cells/cm² in Medium 4.

Day 12: Cells should have reached 100% confluence. Cells were fed with Medium 5.

Day 12-16: Cells were fed daily with Medium 5; at day 16, more than 90% of cells were FOXA2⁺/EN⁺, as measured by FACS analysis.

Day 16-Day 30: Cells were fed daily with Medium 6.

Day 30-Day 100: Cells were fed daily with Medium 7.

Medium 1 composition: neurobasal medium, N2 supplement, B27 supplement, Pen/Strep, L-Glutamine, 10 μM SB, 250 nM LDN, 500 ng/ml SHH C25II, and 1 μM CHIR.

Medium 2 composition: neurobasal medium, N2 supplement, B27 supplement, Pen/Strep, L-Glutamine, 10 mM SB, 250 nM LDN, 500 ng/ml SHH C25II, and 6 μM CHIR.

Medium 3 composition: neurobasal medium, N2 supplement, B27 supplement, Pen/Strep, L-Glutamine, and 6 μM CHIR.

Medium 4 composition: neurobasal medium, B27 supplement, Pen/Strep, L-Glutamine, 1 μM IWP2, 20 ng/ml BDNF, 0.2 μM ascorbic acid (AA), 20 ng/ml GDNF, 0.5 mM dcAMP, and 1 ng/ml TGF-β3.

Medium 5 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, 1 μM IWP2, and 100 ng/ml FGF18.

Medium 6 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, 1 μM IWP2, and DAPT (10 nM).

Medium 7 composition: neurobasal medium, B27 supplement, Pen/Strep, L-Glutamine, 20 ng/ml BDNF, 0.2 μM AA, 20 ng/ml GDNF, 0.5 mM dcAMP, 1 ng/ml TGF-β3, and DAPT (10 nM).

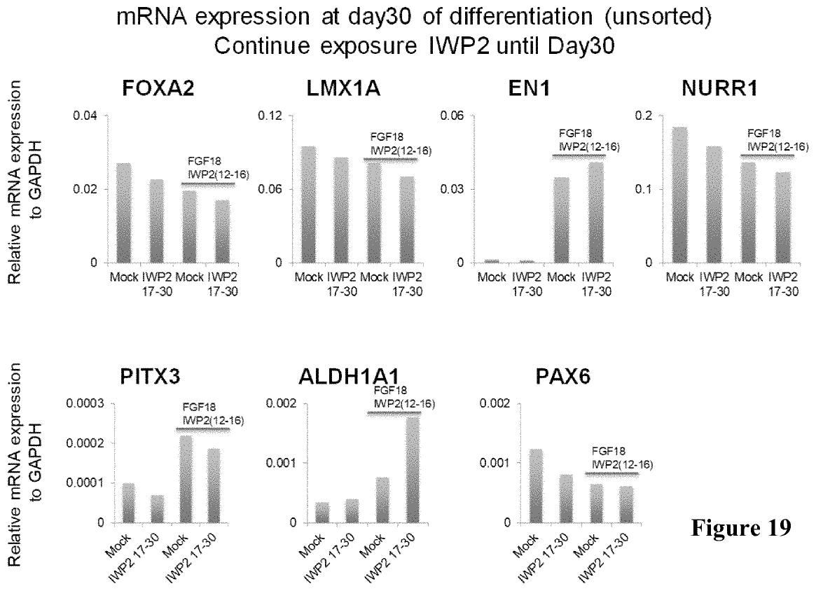

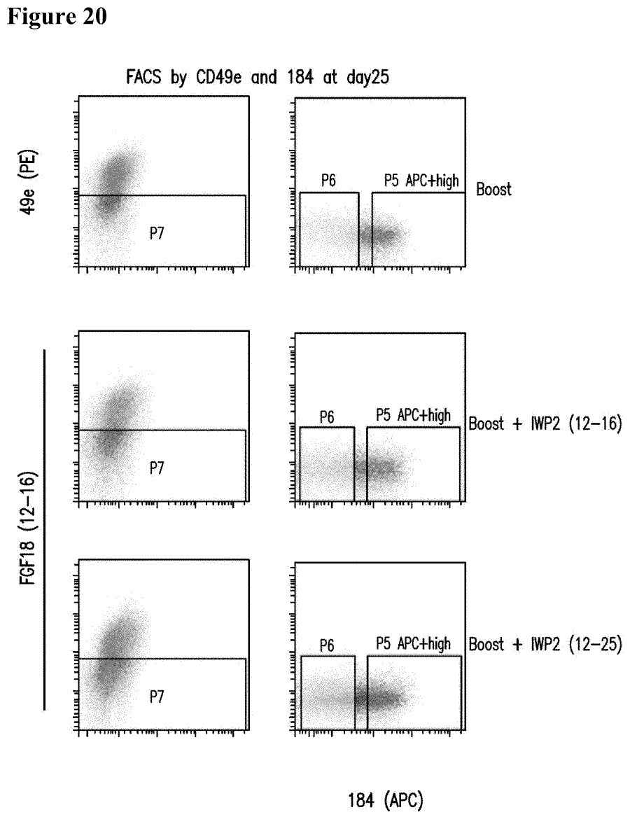

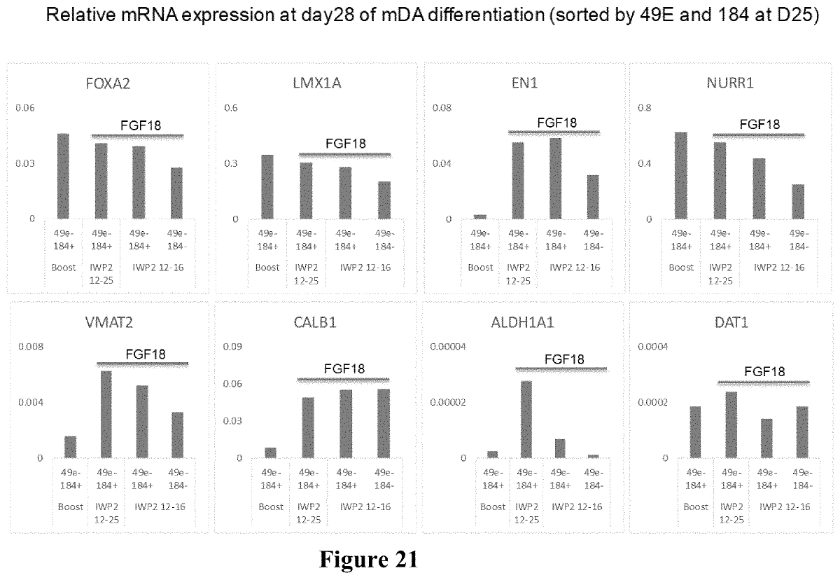

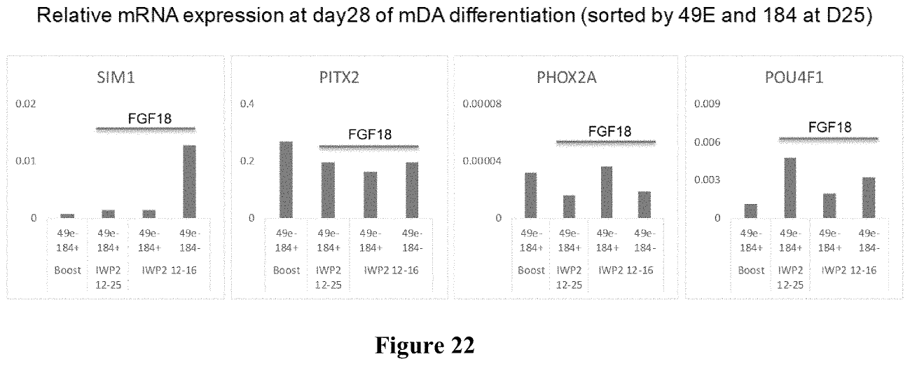

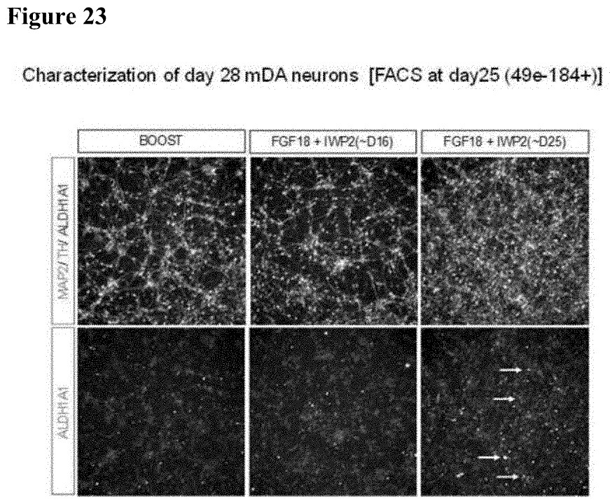

The present disclosure discovered that continued exposure to IWP2 until day 30 further induced the expression of ALDH1A1 (FIG. 19). FIG. 20 showed FACS-mediated sorting strategy of day 25-differentiated cells produced from Wnt-boost protocol with or without the addition of IWP2 from day 12 to day 25, or from day 12 to day 16, and with or without the addition of FGF18 from day 12 to day 16. The mRNA expression of marker genes in sorted day 28-differentiated CD49^{weak}/CD184^{weak} cells and CD49^{weak}/CD184^{strong} cells were measured (FIGS. 21 and 22). Consistent with the results of FIG. 19, exposure to IWP2 from day 12 to day 25 further induced the expression of ALDH1A1. This result was confirmed using immunofluorescent staining (FIG. 23).

### Example 10: In Vivo Transplantation of Differentiated Cells

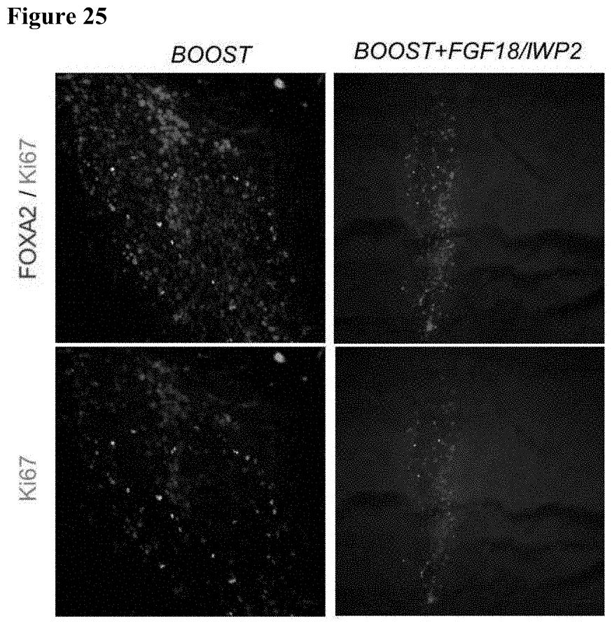

Repeated in vivo transplantation experiments of Example 3 were conducted. Differentiated cells generated according to the Wnt boost with IWP2 and FGF18 protocol had many graft advantages such as improving striatal innervation, maintained EN1 expression, increasing A9 type ALDH1A1⁺ cells, as well as decreasing the number of proliferating cells (Ki67⁺ cells) (FIGS. 24 and 25).

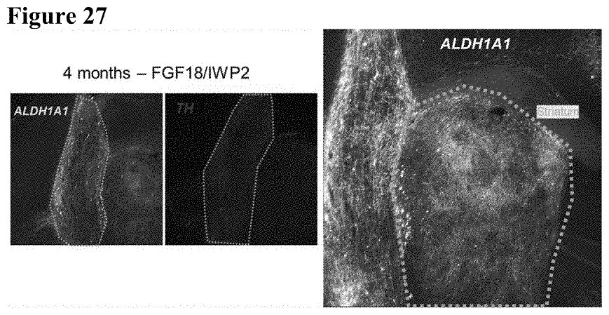

4 months after the transplantation, transplanted cells generated according to the Wnt boost with IWP2 and FGF18 protocol had A9 type DA neurons exonal projection, covering almost only entire striatum regions (FIG. 27).

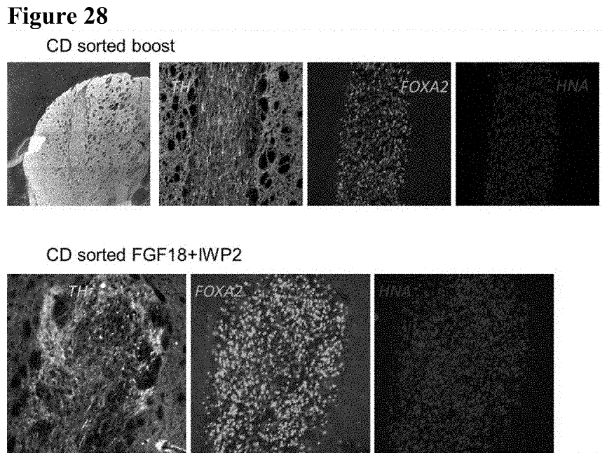

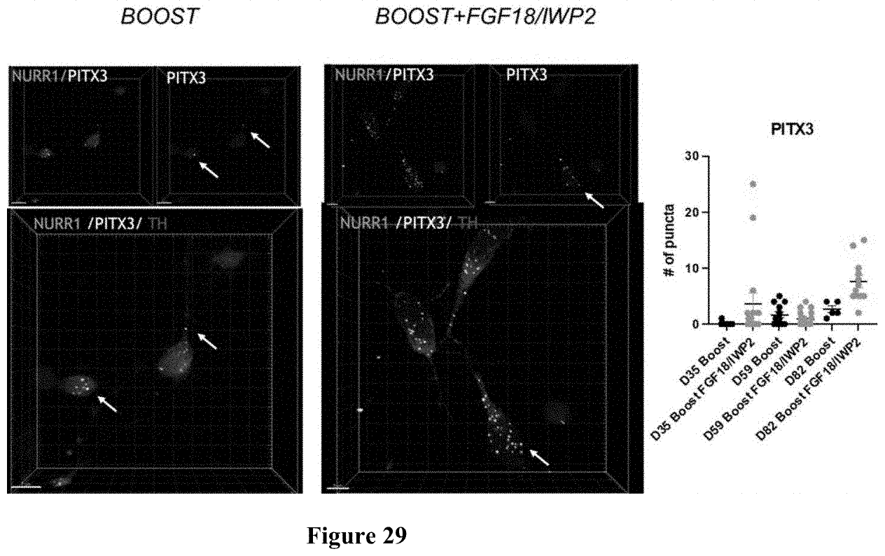

Next, sorted CD49^{weak}/CD184^{strong} cells were transplanted in mouse. Cells were sorted on day 25 of in vitro differentiation under Wnt-boost or Wnt-boost+FGF18/IWP2 (day 12-day 16) protocols. Grafted cells were immune-stained one month after transplantation. The transplanted cells showed good survival and had homogenous DA population expressing TH and FOXA2 in both conditions (FIG. 28). PITX3 expression was also measured in vitro differentiated cells under Wnt Boost with/without IWP2 and FGF18 (day 12-day 16) using RNA in situ assay (FIG. 29).

### Example 11: In Vivo Transplantation of Differentiated Cells

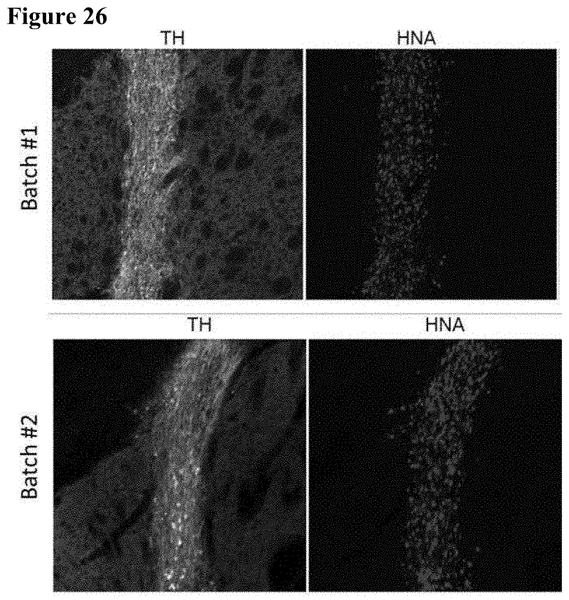

Transplantation of differentiated cells that had been frozen (off the shelf cell source) was examined to test the clinical relevance of the cells generated by the presently disclosed protocols. Two frozen batch cells were transplanted and immune-stained and evaluated by TH and HNA 1-month post graft (FIG. 26). The transplanted cells showed excellent graft survival by expressing mDA markers such as TH and FOXA2 in 2 different batches, demonstrating that the presently disclosed methods are clinically relevant (FIG. 26).

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the present disclosures of which are incorporated herein by reference in their entireties for all purposes.

What is claimed is:

1. An in vitro method for inducing differentiation of stem cells, comprising:
contacting the stem cells with at least one inhibitor of Small Mothers Against Decapentaplegic (SMAD) signaling, at least one activator of Sonic hedgehog (SHH) signaling, and at least one activator of wingless (Wnt) signaling; and
contacting the cells with at least one activator of fibroblast growth factor (FGF) signaling and at least one inhibitor of Wnt signaling to obtain a population of differentiated cells expressing at least one marker indicating a midbrain dopamine neuron or a precursor thereof.

2. The method of claim 1, wherein the contact of the cells with the at least one inhibitor of Wnt signaling is initiated at least about 5 days from the initial contact of the stem cells with the at least one inhibitor of SMAD signaling.

3. The method of claim 1 or 2, wherein the contact of the cells with the at least one inhibitor of Wnt signaling is initiated no later than about 15 days from the initial contact of the stem cells with the at least one inhibitor of SMAD signaling.

4. The method of any one of claims 1-3, wherein the contact of the cells with the at least one inhibitor of Wnt signaling is initiated about 10 days from the initial contact of the stem cells with the at least one inhibitor of SMAD signaling.

5. The method of any one of claims 1-4, wherein the contact of the cells with the at least one inhibitor of Wnt signaling is initiated 10 days, 11 days, 12 days, or 13 days from the initial contact of the stem cells with the at least one inhibitor of SMAD signaling.

6. The method of any one of claims 1-5, wherein the cells are contacted with the at least one inhibitor of Wnt signaling for at least about 1 day.

7. The method of any one of claims 1-6, wherein the cells are contacted with the at least one inhibitor of Wnt signaling for up to about 30 days or up to about 25 days.

8. The method of any one of claims 1-7, wherein the cells are contacted with the at least one inhibitor of Wnt signaling for about 5 days, about 15 days, or about 20 days.

9. The method of any one of claims 1-8, wherein the cells are contacted with the at least one inhibitor of Wnt signaling for 4 days, 5 days, 6 days, 7 days, 14 days, 15 days, 19 days, or 20 days.

10. The method of any one of claims 1-9, wherein the contact of the cells with the at least one activator of FGF signaling is initiated at least about 5 days or at least about 10 days from the initial contact of the cells with the at least one inhibitor of SMAD signaling.

11. The method of any one of claims 1-10, wherein the contact of the cells with the at least one activator of FGF signaling is initiated no later than about 20 days, or no later than 18 days from the initial contact of the cells with the at least one inhibitor of SMAD signaling.

12. The method of any one of claims 1-11, wherein the contact of the cells with the at least one activator of FGF signaling is initiated about 10 days from the initial contact of the cells with the at least one inhibitor of SMAD signaling.

13. The method of any one of claims 1-12, wherein the contact of the cells with the at least one activator of FGF signaling is initiated 10 days, 11 days, 12 days, or 13 days from the initial contact of the cells with the at least one inhibitor of SMAD signaling.

14. The method of any one of claims 1-13, wherein the cells are contacted with the at least one activator of FGF signaling for at least about 1 day and/or for up to about 20 days; at least about 3 days and/or for up to about 10 days; or at least 4 days and/or for up to 7 days.

15. The method of any one of claims 1-14, wherein the cells are contacted with the at least one activator of FGF signaling for about 5 days.

16. The method of any one of claims 1-15, wherein the cells are contacted with the at least one activator of FGF signaling for 4 days, 5 days, 6 days, or 7 days.

17. The method of any one of claims 1-16, wherein the cells are contacted with the at least one inhibitor of SMAD signaling for about 5 days.

18. The method of any one of claims 1-17, wherein the cells are contacted with the at least one inhibitor of SMAD signaling for 6 days or 7 days.

19. The method of any one of claims 1-18, wherein the cells are contacted with the at least one activator of SHH signaling for about 5 days.

20. The method of any one of claims 1-19, wherein the cells are contacted with the at least one activator of SHH signaling for 6 days or 7 days.

21. The method of any one of claims 1-20, wherein the cells are contacted with the at least one activator of Wnt signaling for about 15 days.

22. The method of any one of claims 1-21, wherein the cells are contacted with the at least one activator of Wnt signaling for 16 days or 17 days.

23. The method of any one of claims 1-22, wherein the concentration of the at least one activator of Wnt signaling is increased about 4 days from its initial contact with the stem cells.

24. The method of claim 23, wherein the concentration of the at least one activator of Wnt signaling is increased by between about 200% and about 1000% from the initial concentration of the at least one activator of Wnt signaling.

25. The method of claim 23 or 24, wherein the concentration of the at least one activator of Wnt signaling is increased by about 500% from the initial concentration of the at least one activator of Wnt signaling.

26. The method of any one of claims 23-25, wherein the concentration of the at least one activator of Wnt signaling is increased to from about 1 μM to between about 5 μM and about 10 PM.

27. The method of any one of claims 23-26, wherein the concentration of the at least one activator of Wnt signaling is increased to a concentration of about 6 μM.

28. The method of any one of claims 1-27, wherein the at least one inhibitor of Wnt signaling is capable of inhibiting non-canonical Wnt signaling and canonical Wnt signaling.

29. The method of any one of claims 1-28, wherein the at least one inhibitor of Wnt signaling is selected from the group consisting of IWP2, IWR1-endo, XAV939, IWP-O1, Wnt-C59, IWP-L6, and ICG-001, and combinations thereof.

30. The method of any one of claims 1-29, wherein the at least one inhibitor of Wnt signaling comprises IWP2.

31. The method of any one of claims 1-30, wherein the at least one activator of FGF signaling is selected from the group consisting of FGF18, FGF17, FGF8a, FGF8b, FGF4, FGF2, and combination thereof.

32. The method of any one of claims 1-31, wherein the at least one activator of FGF signaling is capable of causing expansion of the midbrain and upregulating midbrain gene expression.

33. The method of claim 32, wherein the at least one activator of FGF signaling is selected from the group consisting of FGF18, FGF17, FGF8a, FGF4, FGF2, and combination thereof.

34. The method of claim 33, wherein the at least one activator of FGF signaling comprises FGF18.

35. The method of any one of claims 1-34, wherein the at least one inhibitor of SMAD signaling comprises an inhibitor of TGFβ/Activin-Nodal signaling, an inhibitor of bone morphogenetic protein (BMP) signaling, or a combination thereof.

36. The method of claim 35, wherein the at least one inhibitor of TGFβ/Activin-Nodal signaling comprises an inhibitor of ALK5.

37. The method of claim 35 or 36, wherein the at least one inhibitor of TGFβ/Activin-Nodal signaling is selected from the group consisting of SB431542, derivatives of SB431542, and combinations thereof.

38. The method of claim 37, wherein the derivative of SB431542 comprises A83-01.

39. The method of any one of claims 35-38, wherein the at least one inhibitor of TGFβ/Activin-Nodal signaling comprises SB431542.

40. The method of claim 35, wherein the at least one inhibitor of BMP signaling is selected from the group consisting of LDN193189, Noggin, dorsomorphin, derivatives of LDN193189, derivatives of Noggin, derivatives of dorsomorphin, and combinations thereof.

41. The method of claim 35 or 40, wherein the at least one inhibitor of BMP comprises LDN-193189.

42. The method of any one of claims 1-41, wherein the at least one activator of Wnt signaling comprises an inhibitor of glycogen synthase kinase 30 (GSK3β) signaling.

43. The method of any one of claims 1-42, wherein the at least one activator of Wnt signaling is selected from the group consisting of CHIR99021, CHIR98014, AMBMP hydrochloride, LP 922056, Lithium, deoxycholic acid, BIO, SB-216763, Wnt3A, Wnt1, Wnt5a, derivatives thereof, and combinations thereof.

44. The method of any one of claims 1-43, wherein the at least one activator of Wnt signaling comprises CHIR99021.

45. The method of any one of claims 1-44, wherein the at least one activator of SHH signaling is selected from the group consisting of SHH proteins, Smoothened agonists (SAG), and combinations thereof.

46. The method of claim 45, wherein the SHH protein is selected from the group consisting of recombinant SHHs, modified N-terminal SHHs, and combinations thereof.

47. The method of claim 46, wherein the modified N-terminal SHH comprises two Isoleucines at the N-terminus.

48. The method of claim 46 or 47, wherein the modified N-terminal SHH has at least about 90% sequence identity to an un-modified N-terminal SHH.

49. The method of claim 48, wherein the un-modified N-terminal SHH is a un-modified mouse N-terminal SHH or a un-modified human N-terminal SHH.

50. The method of any one of claims 46-49, wherein modified N-terminal SHH comprises SHH C25II.

51. The method of claim 45, wherein the SAG comprises purmorphamine.

52. The method of any one of claims 1-51, wherein at least about 80% of the differentiated cells express FOXA2 and EN1 about 15 days from the initial contact of the stem cells with the at least one inhibitor of SMAD signaling.

53. The method of any one of claims 1-52, wherein greater than about 80% or greater than about 90% of the differentiated cells express FOXA2 and EN1 16 days from the initial contact of the stem cells with the at least one inhibitor of SMAD signaling.

54. The method of any one of claims 1-53, wherein the at least one marker indicating a midbrain dopamine neuron or a precursor thereof is selected from the group consisting of EN1, OTX2, TH, NURR1, FOXA2, PITX3, LMX1A, LMO3, SNCA, ADCAP1, CHRNA4, SOX6, DAT, VMAT2, WNT1, GIRK2, and combinations thereof.

55. The method of any one of claims 1-54, wherein the differentiated cells do no express at least one marker selected from the group consisting of PAX6, EMX2, LHX2, SMA, SIX1, PITX2, SIM1, POU4F1, PHOX2A, BARHL1, BARHL2, GBX2, HOXA1, HOXA2, HOXB1, HOXB2, POU5F1, NANOG, and combinations thereof.

56. The method of any one of claims 1-55, further comprising isolating cells that express at least one positive surface marker and do not express at least one negative surface marker.

57. The method of claim 56, wherein the at least one positive surface marker is selected from the group consisting of CD171, CD184, and combinations thereof.

58. The method of claim 56 or 57, wherein the at least one positive surface marker comprises CD184.

59. The method of any one of claims 56-58, wherein the at least one negative surface marker is selected from CD49e, CD99, CD340, and combinations thereof.

60. The method of any one of claims 56-59, wherein the at least one negative surface marker comprises CD49e.

61. The method of any one of claims 56-60, comprising sorting cells that express CD184 and do not express CD49e.

62. The method of any one of claims 1-61, wherein the stem cells are pluripotent stem cells.

63. The method of any one of claims 1-62, wherein the stem cells are selected from the group consisting of nonembryonic stem cells, embryonic stem cells, induced pluripotent stem cells, and combinations thereof.

64. The method of any one of claims 1-63, wherein the stem cells are human stem cells, non-human primate stem cells, or rodent stem cells.

65. The method of any one of claims 1-64, wherein the stem cells are human stem cells.

66. A cell population of in vitro differentiated cells, wherein the in vitro differentiated cells are obtained by a method of any one of claims 1-65.

67. A composition comprising the cell population of claim 66.

68. The composition of claim 67, which is a pharmaceutical composition further comprising a pharmaceutically acceptable carrier.

69. A kit for inducing differentiation of stem cells to midbrain dopamine neurons or precursors thereof, comprising:
(a) at least one inhibitor of SMAD signaling;
(b) at least one activator of SHH signaling;
(c) at least one activator of Wnt signaling;
(d) at least one inhibitor of Wnt signaling; and
(e) at least one activator of FGF signaling.

70. The kit of claim 69, further comprising (f) instructions for inducing differentiation of the stem cells into a population of differentiated cells that express at least one marker indicating a midbrain dopamine neuron or a precursor thereof.

71. A method of preventing, modeling, and/or treating at least one symptom in a subject having a neurological disorder, comprising administering to the subject an effective amount of one of the followings:
(a) the cell population of claim 66; or
(b) the composition of claim 67 or 68.

72. The method of claim 71, wherein the neurological disorder is characterized by reduction of midbrain dopamine neuron function.

73. The method of claim 72, wherein the reduction of midbrain dopamine neuron function is age related.

74. The method of any one of claims 71-73, wherein the neurological disorder is selected from the group consisting of Parkinsonism, Parkinson's disease, Huntington's disease, Alzheimer's disease, multiple sclerosis, and combinations thereof.

75. The method of any one of claims 71-74, wherein the neurological disorder is selected from the group consisting of Parkinsonism, Parkinson's disease, and combinations thereof.

76. The method of any one of claims 71-75, wherein the symptom for a neurological disorder is selected from the group consisting of tremor, bradykinesia, flexed posture, postural instability, rigidity, dysphagia, and dementia.

77. The cell population of claim 66 or the composition of claim 67 or 68 for use in preventing, modeling, and/or treating at least one symptom in a subject having a neurological disorder in a subject.

78. The cell population or composition for use of claim 77, wherein the neurological disorder is characterized by reduction of midbrain dopamine neuron function.

79. The cell population or composition for use of claim 78, wherein the reduction of midbrain dopamine neuron function is age related.

80. The cell population or composition for use of any one of claims 77-79, wherein the neurological disorder is selected from the group consisting of Parkinsonism, Parkinson's disease, Huntington's disease, Alzheimer's disease, multiple sclerosis, and combinations thereof.

81. The cell population or composition for use of any one of claims 77-80, wherein the neurological disorder is selected from the group consisting of Parkinsonism, Parkinson's disease, and combinations thereof.

82. The cell population or composition for use of any one of claims 77-81, wherein the symptom for a neurological disorder is selected from the group consisting of tremor, bradykinesia, flexed posture, postural instability, rigidity, dysphagia, and dementia.
